(19) United States
(12) Patent Application Publication
(10) Pub. No.: US 2019/0304597 A1
(43) Pub. Date: Oct. 3, 2019

(54) Apparatus or Electronic System for Requisitioning Medical Care

(71) Applicants: Marc A. Grossman, Anaheim Hills, CA (US); Dante Panella, Odessa, FL (US)

(72) Inventors: Marc A. Grossman, Anaheim Hills, CA (US); Dante Panella, Odessa, FL (US)

(21) Appl. No.: 16/254,491

(22) Filed: Jan. 22, 2019

Related U.S. Application Data
(63) Continuation-in-part of application No. 14/918,423, filed on Oct. 20, 2015.
Application No. 14/918,423 is a continuation-in-part of application No. 14/641,066, filed on Mar. 6, 2015, now abandoned.
Application No. 14/641,066 is a continuation-in-part of application No. 14/296,198, filed on Jun. 4, 2014, now abandoned.
(60) Provisional application No. 62/066,809, filed on Oct. 21, 2014.
(60) Provisional application No. 61/876,622, filed on Sep. 11, 2013.
(60) Provisional application No. 61/831,585, filed on Jun. 5, 2013.

(51) Int. Cl. G16H 40/20; G06Q 30/02; G06F 16/27

(52) U.S. Cl. CPC G16H 40/20; G06F 9/45558; G06F 16/27; G06Q 30/0283

(57) ABSTRACT

An apparatus and system for requisitioning medical procedures and/or corresponding devices or services. An apparatus system includes electronic devices or hardware, including processors and storage for containing digital data and transmitting communication signals among local hardware devices and/or remote (e.g., cloud-based) hardware devices in response to stimuli. The system may include particular data structures, variables and/or formats that are matched with stimuli during operation to control system output. Local and remote hardware components may be synchronized for redundancy purposes and/or speed during transactions.

### BACKGROUND

### 1. Field of the Invention

The present invention relates to an apparatus and system for determination of desired medical care or procedures and corresponding ancillary services and the requisition of such services based upon patient desires and/or pre-contracted services or providers. Availability for providers, services, or other auxiliary services may be based upon contracted pricing, determined and/or setup by the apparatus or system, whose information may be made available to a patient or other individual involved in patient care prior to undergoing future medical care, procedures, or services. More particularly, the present invention relates to an apparatus and system that stores or interfaces with data for locating, choosing, comparing costs, comparing or obtaining financing, and booking medical procedures and services via a central server or computerized network based upon parameters input or identified by a user. The cost comparison can include a comparison of in-network medical procedures or services, out-of-network medical procedures or services, cash-only payment by the user, and/or financing options for the medical procedures or services before they are rendered.

### 2. Description of the Related Art

As health care costs in the United States and elsewhere around the world continue to rise, individuals seeking medical treatment are frequently subjected to stress, not only over their immediate medical problems, but also regarding the payment for medical services to treat those very problems. Particularly for individuals that are uninsured, underinsured, or self-insured, the high cost of medical treatment can be a significant hardship. Even for those patients fortunate enough to have a medical care plan or medical insurance plan, cost is still a common concern since many plans require the patient to pay substantial sums of money in the form of high deductibles before the carrier will begin payment on medical bills. Oftentimes, the more affordable a plan is to an individual, the higher the deductible that must be paid.

Compounding these issues is the lack of any price transparency for medical care made available to the general public. Unlike many other service professions which outline, in detail, the cost associated with, the services up-front, medical care is often provided without an itemized cost estimate provided to the patient. Many times, only insurance companies or other health care plans, such as Medicaid or Medicare, have access to the prices of such services. Patients, therefore, have no idea how much a medical procedure will cost when agreeing to it or even when having the service performed. The expense is typically only known once the patient receives the bill or insurance payment statement, sometimes several months after the procedure has been completed, and the cost is often at prices several times higher than usual reimbursements by third party payers to those same providers. Such is die case even for non-emergency medical procedures. The current system deters patients from seeking out desired care from a variety of potential hospitals, outpatient diagnostic/treatment facilities, or medical personnel. Indeed, many individuals or outpatient diagnostic/treatment facilities are not even aware that medical procedure prices are negotiable or that they can vary” wildly even between hospitals that are located in close geographic proximity. This lack of published pricing and the availability of discounts can discourage competition between providers, to the detriment of the patients.

Moreover, because there is often a lack of collateral or secured items in a medical procedure or service, financing can be difficult to obtain for customers through traditional means. Particularly for more expensive medical procedures and/or for cosmetic surgeries, banking or other lending institutions may not desire to provide financial assistance to customers since the risk of such customer failing to pay off the financing and having no secured item to recover in an effort to recuperate those losses can be higher than other loans.

Thus, an apparatus, system, and/or method is desired for reducing one or more of the issues discussed above, such as by allowing prospective patients and/or those working with prospective patients to more adequately obtain desired medical care at prices that are known and/or less costly than traditional medical care costs. An apparatus, system, and/or method may provide prospective patients or those working with prospective patients to more effectively or efficiently shop for preferred health care and/or pay for or finance such health care. The apparatus, system, and/or method may desirably allow potential patients access to prices and costs associated with various procedures to allow those patients to make informed decisions about their health care issues before undergoing a particular procedure. Further, the apparatus, system, and/or method may desirably allow potential patients access to medical care at prices that are reduced from those of traditional medical care.

Moreover, the apparatus, system, and/or method may desirably allow prospective patients, or those working with prospective patients, to conveniently determine information according to the desires of the prospective patient and to easily navigate such information, such as by way of a particular apparatus or system that provides a user interface. In addition, the apparatus, system, and/or method may desirably allow prospective patients, or those working with prospective patients, the option of bundling medical procedures and/or ancillary services for lower pricing and/or more convenient shopping.

### SUMMARY

The present invention is related to an apparatus, system, and/or method for determining and requisitioning medical care. In one embodiment, a system to requisition medical care may include a memory configured to store medical data, the medical data comprising one or more data structures including: a pricing variable that defines an exact price for a medical procedure, and a practitioner variable that defines a practitioner to perform the medical procedure. The system may also include one or more processors connected with the first memory via a network and configured to: receive authorization data, communicate with the first memory via the network to identify approved practitioner data based upon the authorization data, receive request data, communicate with the first memory via the network to locate a medical procedure in the medical data based upon the request data and matching the practitioner variable with the approved practitioner data, and communicate with the first memory via the network to output an exact price for the medical procedure based upon the pricing variable.

In a further embodiment, a system for determining a medical procedure for a user can include a memory configured to store data and a processor connected with the memory. In some embodiments, based at least in part on information received from the user, the processor can be configured to determine a medical procedure, determine a geographic location for the medical procedure, determine a medical facility for the medical procedure at the geographic location, determine a medical practitioner, and determine a price for the medical procedure using the data stored in the memory.

In a further embodiment, a method for providing pricing for medical procedures to a user can include providing a processor and a memory accessible by the processor, receiving, using the processor, user input from the user, determining, using the processor, a medical procedure based on the user input, determining, using the processor, a geographic location for the medical procedure based on the user input, determining, using the processor accessing the memory, a cost for the medical procedure In the geographic location, and displaying, using die processor, the cost to the user.

In a further embodiment, a medical system may include a memory configured to store data and a processor connected with the memory and, based at least in part on information provided by a user. The processor may be configured to determine a medical procedure and determine a price for the medical procedure using the data stored in the memory, the price determined based on at least one of an in-network price for the medical procedure, an out-of-network price for the medical procedure, a copayment price for the medical procedure, a deductible price for the medical procedure, and a cash-only price for the medical procedure.

In a further embodiment, a method for providing pricing for medical procedures may include providing a processor and a memory accessible by the processor, receiving, using the processor, input from die user, determining, using the processor, a medical procedure based on the input from the user, and displaying, using the processor, the cost to the user for the medical procedure, the cost based on at least one of an in-network price for the medical procedure, an out-of-network price for the medical procedure, a copayment price for the medical procedure, a deductible price for the medical procedure, and a cash-only price for the medical procedure.

In a further embodiment, a method of obtaining revenue from a healthcare provider may include providing a processor and a memory accessible by the processor, receiving, using the processor, data associated with at least one of a medical device, a medical procedure, or a medical service offered by the healthcare provider, storing the data in the memory, storing a profile for the healthcare provider in the memory, the profile associated with the data, and associating a keyword with the data stored m the memory,

Some embodiments of the invention may include determining, using the processor, one or more costs of at least one medical procedure or service based on the user input, wherein the cost determination using the processor comprises a comparison of in-network medical procedures or services, out of network medical procedures or services, and cash-only payment by the user for the medical procedures or services before they are rendered.

Some embodiments of the invention comprise a user determining and receiving costs for at least one medical procedure or service through an internet service and/or internet web portal. Some embodiments of the invention include systems, methods, computer program products, and the like, for locating, choosing, and comparing costs, and booking medical procedures and services via a central server or computerized network based upon parameters input or identified by a user. In some embodiments, the internet service and/or internet web portal can comprise at least one cost comparison tool. In some embodiments, the at least one cost comparison tool is configured to enable a user to display the price based at least in part on at least one of a price for an in-network procedure, a price for an out-of-network procedure, a copayment responsibility, a deductible responsibility, an ancillary service cost, and a cash-only payment. In some further embodiments, the price comprises an out-of-pocket cost based at least in part on a met deductible.

In still another embodiment, a method for providing bundled pricing for medical procedures to a user can include providing a processor and a memory coupled with the processor, determining, using the processor, a medical procedure desired by the user, determining, based on data stored in the memory, a predetermined bundle package having a bundle cost, the bundle package including the medical procedure, displaying, using the processor, the bundle package and the bundle cost to the user, and receiving scheduling information from the user to schedule the medical procedure to be performed for the user.

Some embodiments of the invention further comprise the step of providing a software application configured to be executed on a handheld device, and configured to interface with the processor over the Internet for sending the user input to the processor. In some embodiments, the bundle cost includes a cost comparison based on at least one of a price for an In-network procedure, a price for an out-of-network procedure, a copayment responsibility, a deductible responsibility, an ancillary service cost, and a cash-only payment.

In some embodiments of the method, the software application is configured to display at least one webpage. In some embodiments, the at least one webpage includes at least one cost comparison tool. In some other embodiments, the at least one cost comparison tool is configured to enable a user to display the bundled cost based at least in part on at least one of a price for an in-network procedure, a price for an out-of-network procedure, a copayment responsibility, a deductible responsibility, an ancillary service cost, and a cash-only payment. In some embodiments of the method, the bundled price comprises an out-of-pocket cost based at least in part on a met deductible.

In still further embodiments, an enterprise healthcare solution may include a memory’ configured to store data and a processor connected with the memory and, based at least in part on information provided by a user, configured to determine a healthcare procedure or service, determine a price for the healthcare procedure or service using the data stored in the memory, and determine financing for the user based at least partly on the price for the healthcare procedure or service.

Other embodiments may incorporate a method for providing pricing and financing for healthcare procedures or services, the method including providing a processor and a memory accessible by the processor, receiving, using the processor, healthcare input from the user, determining, using the processor, a healthcare procedure or service based on the healthcare input from the user, receiving, using the processor, financing input from the user, and determining, using the processor, a finance information based on the financing input from the user.

In still further embodiments, a method for providing consumer healthcare searching may include providing a processor, providing one or more storage mediums accessible by the processor, receiving, using the processor, procedure data associated with a healthcare procedure or service offered by a healthcare provider, storing the procedure data in at least one of the one or more storage mediums, displaying, using the processor, at least some of the procedure data for selection by a user, receiving, using the processor, financing data offered by a financing institution, and displaying, using the processor, at least some of the financing data for selection by the user.

### BRIEF DESCRIPTION OF THE DRAWINGS

The features, objects, and advantages of the present invention will become more apparent from the detailed description set forth below when taken in conjunction with the drawings, wherein:

FIG. 1 shows a block diagram of a system implementing a price transparency medical procedure search according to one embodiment of the present invention;

FIG. 2A shows a flowchart of a method for implementing a price transparency medical procedure search according to one embodiment of the present invention;

FIG. 2B shows a flowchart of a portion of a method for implementing a price transparency medical procedure search according to one embodiment of the present invention;

FIG. 3 show's a display to a user for a query, the display a part of a system implementing a price transparency medical procedure search according to one embodiment of the present invention;

FIG. 4 show's a display to a user with results of a query, the display a part of a system implementing a price transparency medical procedure search according to one embodiment of the present invention;

FIG. 5 shows a flowchart of a method for implementing bundled pricing in a system implementing a price transparency medical procedure search according to one embodiment of tire present invention;

FIG. 6 shows a display to a user with results of a query and the option of bundled packages, the display a part of a system implementing a price transparency medical procedure search according to one embodiment of the present invention;

FIG. 7 shows a flowchart of a method for implementing financing options in a healthcare procedure or services solution according to one embodiment of the present invention;

FIG. 8 shows a display to a user with results of a financing options query, the display a part of a system implementing a healthcare procedure of services solution according to one embodiment of the present invention;

FIG. 9 shows a flowchart of a method for implementing financing options in a healthcare procedure or services solution according to one embodiment of the present invention;

FIG. 10 shows a data structure for an apparatus for determining or requisitioning of medical care;

FIG. 11 shows an apparatus or system for determining or requisitioning of medical care;

FIG. 12 shows a plurality of data or information stored and used as part of an apparatus or system for determining or requisitioning of medical care; and

FIG. 13 shows a display of an apparatus or system for determining or requisitioning of medical care for use by a prospective patient or individual working with a prospective patient.

### DETAILED DESCRIPTION

The detailed description of some embodiments herein makes reference to the accompanying drawings and pictures, which show the exemplary embodiment by way of illustration and its best mode. While these exemplary embodiments are described in sufficient detail to enable those skilled in the art to practice the invention, it should be understood that other embodiments can be realized and that logical and mechanical changes can be made without departing from the spirit and scope of the invention. Thus, the detailed description herein is presented for purposes of illustration only and not of limitation. For example, the steps recited in any of the method or process descriptions can be executed in any order and are not limited to the order presented. Moreover, any of the functions or steps can be outsourced to or performed by one or more third parties. Furthermore, any reference to singular includes plural embodiments, and any reference to more than one component can include a singular embodiment.

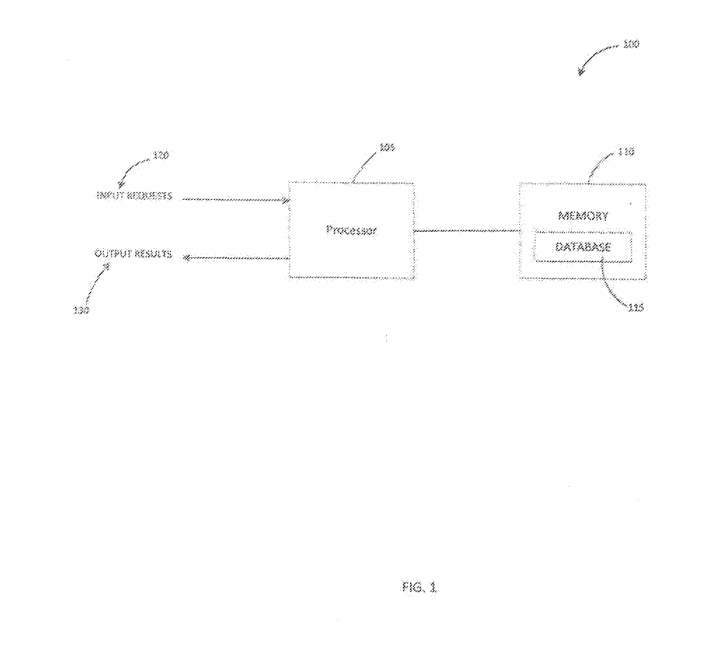

Turning first to FIG. 1, a block diagram of a system 100 implementing a price transparency medical procedure search is shown. As discussed throughout, such a system may be used to provide healthcare information and/or pricing to potential customers (e.g., may be entitled “Healthcare Seeker” or “Healthcare Seeker Technology”). The system 100 includes a processor 105 connected with a memory 110, the memory 110 configured to store data. In some embodiments, the processor 105 is configured to interface or otherwise communicate with the memory 110, for example, via electrical signals propagated along a conductive trace or wire. In an alternative embodiment, the processor 105 can interface with the memory 110 via a wireless connection. In some embodiments, the memory 110 can include a database 115, a plurality of data or entries stored in the database 115 of the memory 110.

As discussed in greater detail herein, in some embodiments, the processor 105 can be tasked with executing software or other logical instructions in order for the price transparency medical procedure search to function as desired. In some embodiments, input requests 120 can be received by the processor 105 (e.g., via signals transmitted from a user at a remote system or device, such as a handheld or mobile device, like a smartphone or tablet, to the processor 105 via a network or internet connection). In an alternative embodiment, the input requests 120 can be received by the processor 105 via a user input device that is not at a geographically remote location (e.g., via a connected keyboard, mouse, etc. at a local computer terminal). In some embodiments, after performing tasks or instructions based upon the user input requests 120, for example, looking up information or data stored in tire memory 110, the processor 105 can output results 130 back to the user that are based upon the input requests 120,

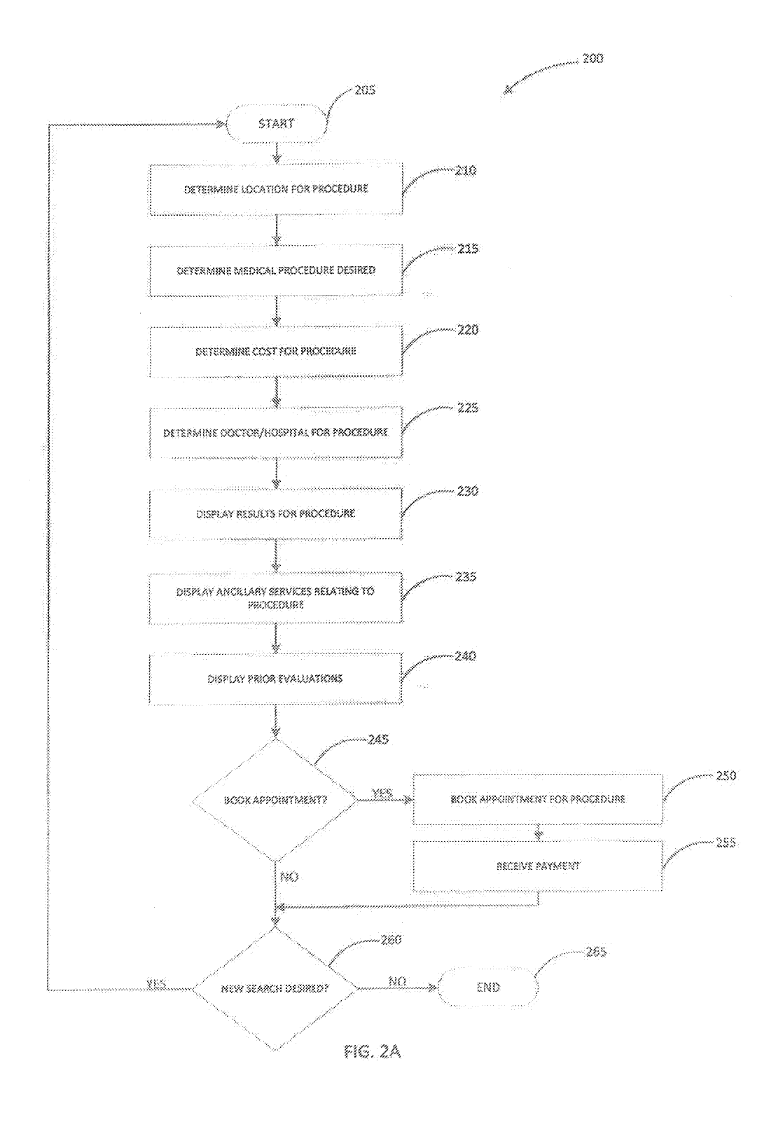

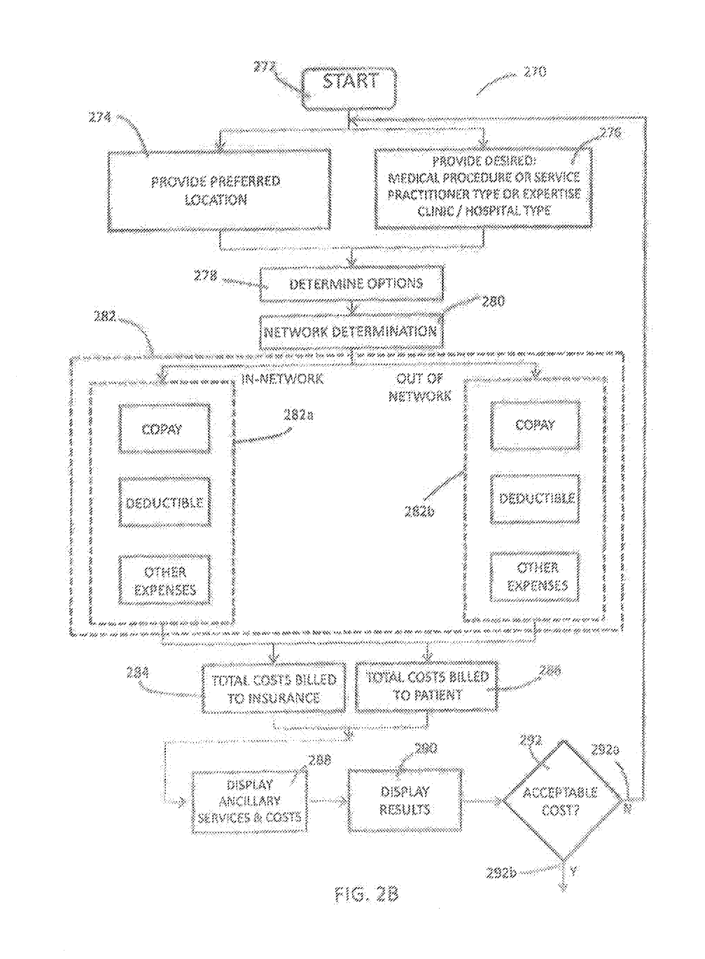

Turning next to FIG. 2A, a flowchart of a process or method 200 for implementing a price transparency medical procedure search is shown. In some embodiments, the process 200 can be implemented in a system that utilizes a processor for evaluating software instructions or code and a memory interfacing with the processor. For example, a system the same as or similar to that described above and shown in FIG. 1 can be used In at least one embodiment of the invention (e.g., utilizing processor 105 and/or memory 110). Thus, in at least one embodiment, a webpage can be established that allows a user to interact with various interactive elements or controls (e.g., drop-down boxes, checkboxes, radio buttons, text boxes, buttons, etc.) in order to determine a desired medical procedure for the user, as discussed in greater detail below. At step 205, the process starts. In one example embodiment, this can occur when a user clicks on a link to arrive at or otherwise enters a webpage URL into an Internet or network browser on their personal computer, tablet, smartphone, etc. Other embodiments can utilize alternative ways for allowing user interaction besides a webpage (e.g., a computer software executable, such as a smartphone or tablet application, etc.)

At step 210, the processor can determine a location or geographic area for a medical procedure that is desired by the user. In some embodiments, this can be determined by examining input from the user that is received via one or more interactive dements or controls placed on a webpage. For example, in some embodiments, a dropdown box or a textbox can be disposed on the webpage and ask for the user to indicate, by selecting or typing, respectively, the geographic area where they would like to have a medical procedure performed. The interactive elements may function by asking the user to indicate a city, state, zip code, address, or the like and can also indicate a desired radius (for example, in miles) that represents the maximum geographic distance from the indicated address that the user would be willing to travel for performance of the medical procedure. In some embodiments, a map can be displayed on the webpage to allow the user to graphically pinpoint the address and desired radius upon the graphical map. For example, a user may be allowed to search in a variety of different countries (e.g., United States, Europe, Canada, etc.) or worldwide,

At step 215, the processor can determine a type of medical procedure that is desired by the user. Similar to step 210, this can be determined by examining input from the user that is received via one or more interactive elements or controls placed on a webpage. For example, a dropdown box or a textbox can be disposed on the webpage and ask for the user to indicate, by selecting or typing, respectively, the type of medical procedure they are interested is having performed. Medical procedures of various fields can be determinable, for example, out-patient surgery, GI Labs, LASIK, dental implants, Medi-Spa, etc.

At step 220, a cost for the desired medical procedure in the location or geographic area is determined by the processor. For example, the processor can interface with a memory that contains a database of locations (e.g., hospitals, physician, or other clinics) that are capable of performing the desired medical procedure. Price data may be stored in the database or obtainable by coupling to other databases or servers, the price data being associated with each of those locations for the desired medical procedure. Thus, based on the determination of a desired medical procedure and a desired location for the procedure in steps 215 and 210, respectively, pricing data can be obtained.

The cost or pricing for the procedure can be on an all-inclusive basis (e.g., can include both facility fees and professional fees). Alternatively, the cost or pricing may be piecemeal (e.g., includes only facility fees, professional fees, or other disparate expenses rather than lumped together). Traditional medical services conventionally do not publish costs to cash paying patients or clients that do not utilize an insurance carrier for substantial portions of the bill. Therefore, using the described system and/or process, price discovery or transparency for medical products or services can be provided to customers where it was traditionally not available. In certain embodiments, additional search criteria related to cost can be available as part of the system and/or process. For example, the form of payment that can be used for paying for the procedure or service may be one additional search criteria. Indeed, any of a variety of search criteria can be used in alternative embodiments, in addition to or alternative to those previously described, for example, price, payment type, geographic location, proximity to related facilities (e.g., rehabilitation centers, etc.), doctor experience, hospital familiarity with the procedure, teaching hospital proximity, etc.

At step 225, the processor can determine hospitals or doctors within the desired area or location that are available to perform the medical procedure desired. This can include hospitals, care facilities, and/or doctors that have signed up with the system of search described by FIG. 2A. For example, membership fees can be paid by such hospitals, care facilities, and/or doctors that wish to be searchable by users utilizing the system. In an alternative embodiment, all hospitals, care facilities, and/or doctors can be included in the search whether or not they have agreed to participate in the system. In still another embodiment, hospitals, care facilities, and/or doctors may agree to be included in the system via pricing that has been negotiated with the owner of the system. Advertising revenue based upon clicks from potential patients to those included hospitals, care facilities, and/or doctors, for example, as discussed in greater detail herein, may be obtained by the owner of the system.

At step 230, the results of the determinations made by the processor in steps 210, 215, 220, and/or 225 for the medical procedure can be displayed to the user. The display of the results can be a single location and cost for the medical procedure desired, and/or can be a list of possible locations and corresponding prices that the user can choose amongst. In this manner, the user can browse the list for deciding the best location and/or cost for having the medical procedure performed. The results can all be displayed at once to the user or can be displayed across various screens or tabs that require the user to click or otherwise manipulate a control on a webpage to see additional results. Some embodiments include a webpage where the results can be organized for display according to various schemes (e.g., from lowest price to highest price, from closest location to furthest location, etc.). In certain embodiments, the user can choose or manipulate how the results are organized for display. In another embodiment, pricing can be determined in step 220 for locations outside of the location determined in step 210, and the user can be prompted as to whether they would desire to see additional results that extend beyond the location previously determined. Further, in some embodiments, a user may choose to accept costs based on one or medical providers where at least a portion of the cost is covered by medical insurance for the user and/or at least a portion of the cost being paid by cash or otherwise out of pocket by the user. In some instances, the user can select to pay at least a portion of the cost even though that portion may be covered by a medical insurance policy covering the user. In other instances, the user can select to pay at least a portion of the cost that may not be covered by a medical insurance policy. In other embodiments, the user can select to pay all of the costs that are displayed in step 230.

At step 235, ancillary services relating to the medical procedure can be displayed to the user. For example, a medical procedure that would require lengthy patient care after surgery, such as physical training to re-learn muscle movements, can display memberships to nearby physical training centers feat the patient can choose to add to the price of the medical procedure. For example, the user may choose to accept ancillary services costs based on one or more medical providers where at least a portion of the ancillary services cost is covered by medical insurance for the user and/or at least a portion of fee ancillary services cost is being paid by cash or otherwise out of pocket by the user. In some instances, the user can select to pay at least a portion of fee ancillary services cost even though that portion may be covered by a medical Insurance policy covering the user. In other instances, the user can select to pay at least a portion of the ancillary services cost that may not be covered by a medical insurance policy. In other embodiments, the user can select to pay all of the ancillary services costs that are displayed in step 235.

Any of a variety of ancillary services can be displayed that would be relevant, to the user considering the medical procedure. In some embodiments, the display of these ancillary services can be on the same page or screen as the results displayed according to step 230. Alternatively, or additionally, the display of these ancillary services can be segregated onto a separate link, page or screen for the user to browse if desired. In some embodiments, providers of such ancillary services can sign up with the system with pricing at a discounted rate. In this fashion, both the operator of the system benefits (in die form of a greater number of providers offering services at low prices) and the providers themselves benefit (in the form of increased exposure to potential customers using the system),

At step 240, one or more prior evaluations for the medical procedure (at the location determined in step 210 and/or the hospital or doctor in step 225 and/or in general) can be displayed to the user. Further, in some embodiments, if multiple locations, hospitals, or doctors are displayed to the user in step 230, prior evaluations can be shown to the user, if existing, for each of the display results. These prior evaluations may be aggregated, user-submitted evaluations from prior patients that had the medical procedures performed at those locations, hospitals, or doctors. Similar to the discussion above for step 230, the order or organization of the prior evaluations can initially be determined by the processor (e.g., newest evaluations first), but can be modified by the user to have a different organization if desired (e.g., most critical or negative evaluations first). In certain embodiments, users may be able to rate prior evaluations, such as by clicking a link Indicating that a particular evaluation was helpful to read. In such embodiments, users may be able to sort or modify the evaluations according to most helpful to least helpful,

In some embodiments, the prior evaluations can be factual data, reviews, data, qualifications, and/or statistics according to other sources, such as the number of times a doctor has performed a particular medical procedure, the number of patients a hospital treats in a year, where a particular doctor went to medical school, etc. Any of a variety of different information can be shown to the user for the purposes of providing data about the individuals or facilities involved in the performance of the medical procedure to aid the user in choosing their preferred doctor, hospital, and/or other metric. For example, one user may be more concerned about price over any other aspect, while another user may be willing to pay a higher price for a doctor with greater experience in performing the type of medical procedure of interest, or to be seen by a doctor at a preferred facility and/or location.

In certain embodiments, the reviews, data qualifications, and/or statistics as mentioned above can be used in order to determine which search results and/or in which order those results will be displayed to the user in step 230, For example, prompts, queries, or other controls that allow' for user manipulation can allow, a user to receive information or results more suited to that user's particular interests for aiding in making an informed choice. For example, a user may choose to only see results for doctors above a certain experience level (e.g., number of years practicing, number of procedures performed, etc.) even if such results yield higher costs than would otherwise be displayed. In another example, a user may desire to browse the lowest costs for a medical procedure, but only for those procedures using a doctor above a certain experience level. Similarly, a user may desire to browse the lowest costs for a medical procedure, but only for those procedures at a hospital that have a particular number of those same procedures performed within over a particular period of time. Any of a number of search criteria can be included such that the user may decide which search criteria to limit the search according to those aspects which are most important to them.

At step 245 the system including the processor can determine whether the user wishes to book an appointment for one of the results displayed in step 230. This can occur, for example, in response to the user selecting one of the results displayed and clicking on a link indicating a desire to schedule the medical procedure per the selected result. If an appointment is desired, operation can continue to step 250, If no appointment is desired, operation can continue to step 260, discussed in greater detail below. At step 250, available times and/or dates for the medical procedure at the selected result can be displayed for the user to browse and choose a desired appointment time. In some embodiments, the user may be able to select a desired span of dates or times, and the processor will determine and/or cause to display available appointment times within that span. At step 255, the processor can receive payment information (e.g., a credit card number, a PayPal® account a deposit account number, etc.) from the user to pay for all or a portion of the medical procedure. In some embodiments, no payment may be required. PayPal® and the PayPal logo are registered trademarks of PayPal, Inc.

Operation can then continue to step 260 to determine whether a new medical procedure search is desired. This can occur, for example, in response to user input indicating they wish to modify a current or already existing search that they have performed or whether they wish to begin a new′ search from the beginning. If no new search is desired, operation continues to step 265 where the process ends. However, if a new search is desired, operation can continue back to step 205 where the process starts anew. Alternative embodiments can utilize additional or fewer steps than those explicitly shown in the flowchart of FIG. 2A. Certain steps can be combined. Certain steps can be omitted. Additional steps may be inserted. Steps can additionally or alternative be performed in a differing order from those explicitly shown and described herein.

In some embodiments of the invention, the cost for any medical procedure or medical service in a specified location or geographic area (for example, specified by a processor, such as processor 105 or by the patient) can be computed by the processor based on a variety of parameters in addition and/or alternatively to those described earlier. Further, in some embodiments, the cost data computed by the processor can be displayed to a patient in a variety of ways to assist the patient in making an informed decision to proceed with any chosen medical procedure or medical service. For example, the cost for the desired medical procedure or service (e.g., represented by step 220 in FIG. 2A) can be processed by the processor using one or more of the steps illustrated in the flowchart 270 shown in FIG. 2B. Further, in some embodiments, steps 205-240 of the process or method 200 shown in FIG. 2A can be substituted by the steps and methods shown in FIG. 2B (the steps forming the flowchart 270).

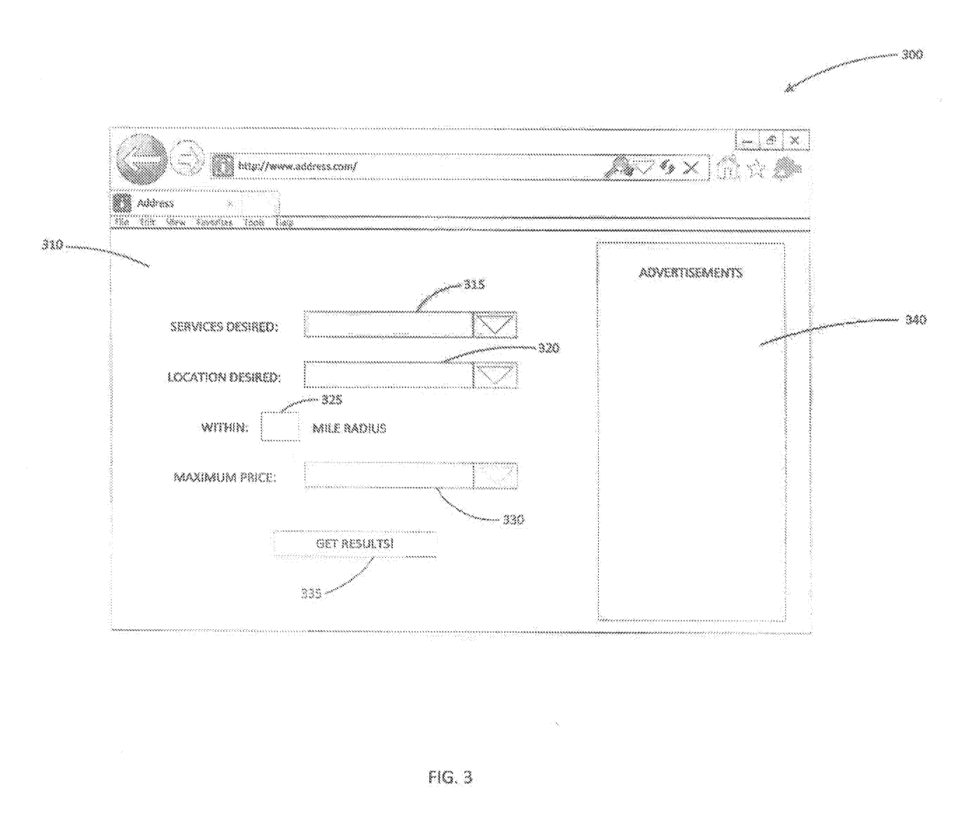

Turning to flowchart 270 of FIG. 2B, and with reference to FIG. 3, in some embodiments of the invention, at step 272, the process starts. In one example, this can occur when a user clicks on a link to arrive at or otherwise enters a webpage URL into an Internet browser on their personal computer, tablet, smartphone, etc. Other embodiments can utilize alternative ways for allowing user interaction besides a webpage (e.g., a computer software executable, such as a smartphone or tablet application, etc.).

At step 274 a patient can be enabled to enter (e.g., select from a drop-down menu and/or web-enabled map) a desired geographic location for the medical procedure or service (e.g., a geographic location where the patient wishes to receive the medical treatment or service). For example, FIG. 3 shows a display 300 that can be presented to a user for a query, where the display 300 comprises a part of a system Implementing a price transparency medical procedure search. In some embodiments, the system can include features that are the same as or similar to those previously discussed. For example, in some embodiments, the display 300 can be part of a system that allows a user to search for information regarding a desired medical procedure, as discussed in relation to the embodiments of FIGS. 1, 2A and/or 2B, As illustrated, some embodiments of the invention can include a display 300 that can comprise a webpage that is accessed using a standard Internet browser (e.g., Internet Explorer®, Google Chrome®, Mozilla Firefox®, etc.) and includes content 310 that is displayed in the browser, Internet Explorer® is a registered trademark of the Microsoft Corporation in the United States and/other countries. Firefox® is a registered trademark of the Mozilla Foundation. Google Chrome is a trademark of Google Inc.

In some embodiments, a dropdown box or a textbox can be disposed on the webpage with a query for the user to indicate, by selecting or typing, respectively, the geographic area and/or location where they would like to have a medical procedure or service performed or provided, as discussed in greater detail herein for FIG. 3. The interactive elements can function by asking the user to indicate a city, state, zip code, address, or the like and can also Indicate a desired radius (for example, in miles) that represents the maximum geographic distance from the indicated address that the user would be willing to travel for performance of the medical procedure.

A step 276 can enable the patient to enter a desired medical procedure or service. In some embodiments of the invention, die display 300 can include a first dropdown box 315 that can allow a viewer of the display 300 to choose a desired medical service or procedure from a pre-generated list. In some alternative embodiments of the Invention, other forms of controls or elements in place of or in addition to the first dropdown box 315, or any of the other controls or elements discussed herein can be used for allowing a user to indicate a desired medical service or procedure.

Like mentioned above, in some embodiments, the display 300 can include a second dropdown box 320 that allows the viewer of the display 300 to choose a desired location for the medical service or procedure to be performed from a pre-generated list. In some embodiments of the invention, a textbox 325 can enable the viewer to farther indicate a desired distance from the chosen location that would be desirable. Further, in some embodiments, other forms of controls or elements in place of or in addition to the second dropdown box 320 and/or the textbox 325 can be used for allowing a user to indicate a desired location. In some embodiments, a map can be displayed on the webpage to allow the user to graphically pinpoint the address and desired radius upon the graphical map. Certain embodiments can allow a user to search in a variety of different countries (e.g., United States, Europe, Canada, etc.) or worldwide. Once a patient has made a selection, the processor can examine input from the user that is received via one or more interactive elements or controls placed on a webpage.

In some embodiments, the patient can be presented with an option to select a type of medical practitioner (such as a family doctor, an internist, a nurse practitioner, a specialist and/or surgeon, etc.). In some further embodiments, the step 276 can also enable a patient to specify a type of medical facility or dime. For example, a patient can select a family clinic, an emergency clinic, a general hospital, an emergency hospital, and/or surgery, etc.

At step 274, the processor can determine a location or geographic area for a medical procedure that is desired by the user (as selected in during step 276 described above) based at least in part on the information provided by the patient in step 276, For example, the processor can interface with a memory that contains a database of locations (e.g., hospitals, physician, or other clinics) that are capable of performing the desired medical procedure. In some embodiments, the medical procedure or service can be available near a geographic location preferred by the patient, in other embodiments, the medical procedure or service can be available some distance horn the geographic location preferred by the patient. In some embodiments, when presented with a plurality of choices, using web-page enable procedures similar or substantially the same as described above with respect to the embodiments depicted in FIG. 3, tire patient can be presented with an option to choose one or more option based on geographic preference. For example, a patient can select at least one choice based on how close the medical procedure or service provider is to their home or office or other desired geographic location. Certain patients may be willing to travel to a specific geographic location base on preference, personal or business travel, or other factors important to the patient,

In some other embodiments of the invention, the preferred location (for receiving or accessing a medical procedure or service) can default to the home of the patient, a business or place of work of the patient, a home, work, or business of a relative of the patient, or the patient's actual location (e.g., the current location of the patient while interacting with the system). In certain embodiments, the patient's location can be determined based at least in part on the location of device used to display and interact with the patient. For example, the device location can be determined by an IP address. In another example, the device location can be determined by a GPS coordinate. In still another example, the device location can be determined by at least one wireless signal from the device.

Depending at least on the information provided by the patient and/or information processed by the processor during performing the method comprising step 274 and/or step 276, one or more potential medical delivery options can be determined by the patient and/or the processor. For example, at step 278, in some embodiments, the processor can determine medical delivery options within the desired, selected, or calculated area or location that are available to perform the medical procedure desired. These options may be further determined in conjunction with step 280, as discussed in greater detail below,

In some embodiments, these medical delivery options can include hospitals, care facilities, dimes, doctors, and/or specialists that have signed up with the system of search described by FIG. 2B. In some embodiments, membership fees can be paid by such hospitals, care facilities, clinics, doctors, and/or specialists that wish to be searchable by users utilizing the system and methods described herein. In an alternative embodiment, all hospitals, care facilities, clinics, doctors, and/or specialists can be included in the search whether or not they have agreed to participate in the system and methods described herein. In still another embodiment, hospitals, care facilities, and/or doctors may agree to be included in the system via pricing that has been negotiated with the owner of the system. Advertising revenue based upon clicks from potential patients to those included hospitals, care facilities, and/or doctors, for example, as discussed in greater detail herein, may be obtained by the owner of the system.

At step 280, the processor determines whether a desired medical procedure or service would be considered in-network or out-of-network for the user. An in-network medical provider can include health care providers and facilities that have fee agreements in place with one or more health insurers. These providers are considered in network for members (e.g., a patient insured through a member network, and, in general, paying lower copayments and deductibles for those member network services). An out-of-network medical provider can include health care providers who do not have an agreement with a health insurer. These providers are generally considered out-of-network, and patients who select and receive medical care from an out-of-network provider or facility do not have the advantage of a negotiated rate, and services rendered may be more expensive and/or may not be completely covered the patient's health plan.

Other aspects of the patient's financial responsibility can also be affected by whether the healthcare provider is in-network or out-of-network. For example, a patient's copayment or “copay” can depend on whether the healthcare provider is considered in-network or out-of-network. In the United States, a copayment is typically a payment defined by a medical insurance policy, and is generally paid by the insured person each time a medical service is provided. Depending on the patient's health insurance plan, a copay may or may not be applied to the patient's deductible. The patient's deductible can also be affected by whether the healthcare provider is in-network or out-of-network, and is generally lower for in-network providers offering comparable care.

Other expenses that can also be affected by whether the healthcare provider is in-network or out-of-network can include a coinsurance payment (such as a percentage payment after the patient's deductible up to a defined limit). Typically, the coinsurance payment must be paid before my policy benefit is payable by an Insurance company, and is applied towards any policy out-of-pocket maxima.

This determination of step 280 may be based upon input received from the user (e.g., response to a query asking for information about the user's medical insurance and/or lookup of what services and/or procedures are considered in-network and/or out-of-network based upon that user's medical insurance). At step 278 the processor can determine and generate in-network and/or out-of-network medical delivery options based at least in part on information provided by a patient and/or generated by the processor in step 274 and/or step 276 and/or step 280.

As illustrated in FIG. 2BS following completion of step 280, the processor can determine in-network and out-of-network costs based at least in part on the healthcare provider information from steps 274, 276, 278, 280. For example, the processor can determine costs from any in-network providers (shown as step 282a). In some other embodiments, costs can be determined by any out-of-network providers (shown as step 282b), In some embodiments, one or more costs calculated in either of the steps 282a and 282b can comprise copay costs, deductible costs, other expense costs, and/or a combination thereof. In some embodiments, the out-of-pocket expenses can comprise costs calculated by the processor in either of steps 282a, 282b that are based on the patient's copay and/or the patient's deductible. For example, in some embodiments, the costs displayed can be adjusted based on a patient's copayment responsibility. In some further embodiments, die costs displayed can be adjusted based on a patient's deductible responsibility. In some other embodiments, the costs displayed can be adjusted based on a patient's copayment responsibility and deductible responsibility. In some embodiments, any adjustments to the costs can be based on the patient's prior payment history. For example, an adjustment can be made based on the patient's remaining deductible that comprises an adjusted deductible based on at least one previous medical procedure or service.

In some embodiments, the other expense cost of step 282a and/or 282b can include the aforementioned coinsurance payments, other out-of-pocket expenses, and/or any other expense that the patient can incur that is not covered as part of his or her copay and/or deductible expense. For example, following completion of step 280 or step 278, the processor can determine cash-only costs based at least in part on the healthcare provider information from at least one of the steps 274, 276, 278, and/or 280. In some embodiments, a user (such as a patient) can be an insured and at least partially covered for medical benefits, but may want to compare total costs by comparing the insurance covered cost (or insurance covered portion of the cost) with a cash payment cost that can Include instances where the patient pays up to 100% of the cost.

For example, the processor can determine cash-only costs from any in-network providers (shown as step 282a), In some other embodiments, cash-only costs can be determined for any out-of-network providers (shown as step 282b), In other instances, a user may be uninsured, and therefore will need to compare total costs for cash-only payment costs. In some other instances, a user may be uninsured, about to be insured, or in a position to receive future medical insurance, and may want to compare total costs by comparing the insurance covered cost (or insurance covered portion of the cost) with a cash payment cost that can include instances where the patient pays up to 100% of the cost. For example, an uninsured user may want to review medical procedures and services in advance of any future medical Insurance coverage, so as to be provided with the option of moving forward with one or more medical procedures or appointments prior to receiving medical insurance, or to delay moving forward with receiving a medical procedures or service prior until die user is at least partially insured for the medical procedures or service.

In some embodiments of the invention, healthcare provider information determined by the processor from step 280 can comprise only in-network providers. In some other embodiments of the invention, the healthcare provider information determined by the processor from step 280 can comprise only out-of-network providers. In some further embodiments, the healthcare provider information determined by the processor from step 280 can comprise a combination of in-network providers and out-of-network providers. For example, in some embodiments, depending on information from any one or combination of steps 274, 276, and/or 278, the medical delivery can comprise healthcare delivery that comprises an in-network and an out-of-network delivery′ category.

For example, the healthcare can comprise more than one service and/or procedure, at least one of which is an in-network procedure or service, and at least of which is an out-of-network procedure or service. A component of the medical delivery might comprise being attended by an out-of-network doctor in an in-network medical facility, or vice-versa. Alternatively, in some embodiments, the medical delivery might comprise being attended by one or more out-of-network doctor for at least one component or phase of the medical procedure or service, and one or more in-network doctors for at least one other component, portion, or phase of the medical procedure or service.

In some embodiments of the invention, following the processor calculating one or more costs in step 282 comprising (comprising steps 282a and/or steps 282b), the processor can calculate total costs and display those calculated costs to the patient. In some embodiments, the processor can use price data for copayments, deductibles, in-network expenses, out-of-network expenses, and other expenses as described above, and a combination thereof stored in a database or obtainable by coupling to other databases or servers. Moreover, in some embodiments, the processor can use price data and display costs for cash-only payments. The price data can be associated with each of location, facility, doctor or specialist, and ancillary staff for the desired medical procedure.

The cost or pricing for the procedure can be on an all-inclusive basis (e.g., include both facility fees and professional fees). Traditional medical services conventionally do not publish costs to cash paying patients or clients that do not utilize an insurance carrier for substantial portions of the bill. Using the described process, price discovery or transparency for medical products or services can be provided to customers where it was traditionally not available, including cash-only costs, in addition to partially or fully covered in-network and out-of-network provider costs. In certain embodiments, additional search criteria related to cost can be available as part of the system, for example, the form of payment that can be used for paying for the procedure or service. Indeed, any of a variety of search criteria can be used in alternative embodiments, in addition to or alternative to those previously described, for example, price, payment type, geographic location, proximity to related facilities (e.g., rehabilitation centers, etc.), doctor experience, hospital familiarity with the procedure, teaching hospital proximity, etc.

At step 284, the processor can calculate one or more total costs covered or payable by one or more medical insurers. In some embodiments, the costs may be spread across multiple insurers or a single insurer. In some embodiments, the total cost calculated at step 284 can represent a portion of the total costs calculated in step 282. In some other embodiments, the total cost calculated at step 284 can represent all of the total costs calculated in step 282. Further, in some embodiments, at step 286, the processor can calculate one or more total costs billable to or payable by the patient. The costs calculated by step 284 represent the total cost responsibility of the patient for the medical procedure or service determined in step 276. In some embodiments, the total cost calculated at step 286 can represent a portion of the total costs calculated in step 282. In some other embodiments, the total cost calculated at step 286 can represent all of the total costs calculated in step 282. In some further embodiments, the costs calculated by step 284 can be zero, and the total cost calculated at step 286 can represent 100% of the total cost of the medical procedure or service. For example, this can occur if the procedure Is not covered by the user's medical insurance plan, or if the user has chosen a cash-only option.

At step 286, the one or more total costs billable to or payable by the patient can comprise costs calculated by the processor in either of steps 282a, 282b that arc based on the patient's copay and/or the patient's deductible. For example, the costs displayed at step 286 can be adjusted based on a patient's copayment responsibility. In another example, the costs displayed at step 286 can be adjusted based on a patient's deductible responsibility. In still another example, the costs displayed at step 286 can be adjusted based on a patient's copayment and deductible responsibility.

In some embodiments, any adjustments can be based on the patient's prior payment history. For example, an adjustment can be made based on the patient's remaining deductible that comprises an adjusted deductible based on at least one previous medical procedure or service. Further, the costs displayed at step 286 may include a comparison of cash-only payment costs compared with out-of-pocket expenses that can comprise copayment costs and/or deductible costs. Further, in some embodiments, the costs displayed at step 286 may include a comparison of cash-only payment costs compared with out-of-pocket expenses that can comprise copayment costs and/or deductible costs.

In some further embodiments, at step 288, ancillary services relating to the medical procedure or service can be displayed to the user. As described earlier with respect to step 235 of FIG. 2A, any of a variety of ancillary services can be displayed that can be relevant to the user considering the medical procedure or service. At step 290, the results of the determinations made by the processor in step 290, and including data from any one or combination of the steps 274, 276, 278, 280, 282, 282a, 282b, 284. 286, and/or 288 for the medical procedure or service can be displayed to the user (such as a patient). In some embodiments, the display of these ancillary services calculated by die processor can be on the same page or screen, for example, as shown displayed according to step 230 of FIG. 2A.

In some embodiments, die display of the results can be a single location and cost for the medical procedure or services desired, selected, or calculated in steps 276, or can be a list of possible locations and corresponding prices that the user can choose amongst. Further, in some embodiments, the user can view cost sub-totals including copayment, deductible, other expenses, etc., as well as view costs displayed for any combination of in-network and out-of-network provider, or for cash-only options. In another embodiment, the user can view a pool of providers and their location, and costs based on in-network, out-of-network, and cash-only payment can be displayed. In this manner, the user can browse the list for deciding the best location mid/or cost for having the medical procedure performed. The results may all be displayed at once to the user or, in other embodiments, can be displayed across various screens or tabs that require the user to click or otherwise manipulate a control on a webpage to see additional results. Some embodiments may include a webpage where the results can be organized for display according to various schemes (e.g., from lowest price to highest price, from closest location to furthest location, etc.). In certain embodiments, the user can choose or manipulate how the results are organized for display.

In some further embodiment, the pricing determined in step 290 can be adjusted based on further selection or preferences from the user. For example, in some embodiments, a user can be referred back to any of the steps 274 and/or 276 to modify or change the location and/or any aspect or component of the originally selected or calculated medical procedure or service. In this manner, tire results displayed by step 290, as calculated in steps 282, 282a, 282b, 284, 286, and/or 288 can be substantially dynamic based at least in part on one or more additional selections or input from a user. Further, in some embodiments, any results displayed by step 290 can change at any time based on any change to any price data in any database or memory used by the processor.

In some embodiments of the invention, following review of any of the results displayed by step 290, the user can be prompted as to whether they would desire to see additional results that extend beyond the location previously determined. For example, the user can be prompted as to whether they would like to add any additional medical services or procedures, and/or modify any medical services or procedures. In some embodiments, as new information is entered, the processor can dynamically update the displayed cost information in step 290, and/or the user can select to update the cost information at any time.

In some embodiments of the invention, a user can provide updated information in steps 274 and/or 276 based on preferences for one or more in-network or out-of-network providers. In some embodiments, a user can attempt to modify costs displayed by fee step 290 by updating input provided to either or both of steps 274 and/or 276, For example, in some embodiments, based at least in part in location information received in step 274, the options determined in step 278 may include only one or more out-of-network providers. In this instance, a user may not wish to use an out-of-network provider and may wish to change information provided in steps 274 or 276, or both. In some embodiments, in decision step 292, if a user decides any of the costs displayed by step 290 are not acceptable, the user can choose a costs not acceptable decision path 292a, and the systems and methods represented by the flowchart 270 can move back to steps 274 and/or 276. In some other embodiments, the user may decide the costs are not acceptable based on any of costs in steps 282a and/or 282b, including, but not limited to copayment cost, deductible cost, cash-only costs, and other expenses associated with the medical procedure or service. Further, in some embodiments, a user can decide on costs not acceptable decision path 292a based at least in part on ancillary costs displayed in step 288 and/or 290.

In some embodiments, the user can select acceptable ranges of costs. For example, referring again to FIG. 3, the display 300 also includes a third dropdown box 330 that allows a viewer of the display 300 to choose a maximum price and/or a price range the viewer would be willing to pay for performance of the medical service or procedure at the location indicated. The third dropdown box 330 (or other, similar dropdown boxes or other input mechanism) can enable a viewer of the display 300 to choose a maximum price the viewer would be willing to pay for an in-network and/or an out-of-network procedure or service. In some other embodiments, the dropdown box 330 (or one or more additional or alternative dropdown boxes) can enable a user to select a similar maximum price and/or a price ranges for copayments, deductibles, other expenses, and any ancillary expenses.

In m alternative embodiment, other forms of controls or elements in place of or in addition to the third dropdown box 330 can be used for allowing a user to indicate their maximum or range of desired price. For example, a button 335 can be clicked by the viewer upon selection or entering of information according to elements 315, 320, 325, and/or 330 to send the selected information to a processor or other component of the system. In certain embodiments, not all of the information shown in FIG. 3 need be submitted or entered before choosing to run a search for results. This information can then be used for determining search results for subsequent display to the viewer,

In some embodiments of the invention, the system can display one or more commercial banners or other advertisements. For example, referring again to FIG. 3, in some embodiments of the invention, advertisements 340 can be shown on the display 300 for providing a source of revenue for the system implementing the display 300. The revenue from advertisements 340 may be the only form of revenue for the system or other streams can be received in conjunction with or in replacement of advertisements 340 in alternative embodiments. For example, participating facilities that perform the medical procedures that are capable of being searched (e.g., hospitals, surgery centers/providers, etc.) can pay an initial membership fee and/or an ongoing monthly maintenance fee to be included as part of the system. In still another example, a participating facility can additionally or alternatively pay a search fee and/or or a click-through fee when a medical procedure is performed due to a searched or scheduled appointment from using the system implementing the display 300.

As previously mentioned, advertising fees from patient click-through may provide revenue (e.g., sole revenue or in combination with other revenue generating schemes) for the owner of the system implementing the display 300 (or any of the other possible embodiments discussed throughout). For example, the system may be a platform that allows physicians, healthcare facilities, or other medical providers or care centers to create or allow creation of a profile on the system that lists certain devices, procedures and/or services available. These devices, procedures and/or services listed on the healthcare provider profile may be the same as those discussed herein that are available for potential customer selection and booking. In this manner, in addition or alternatively to the results shown to die user based upon user input previously discussed, a user may also be permitted to visit the profile of a desired healthcare provider (e.g., if a user inputs a specific hospital or geographic region, the results shown to the user by the system may include links to the profile page for such corresponding facilities and include procedures, services, etc. and die costs associated therewith).

This profile and/or the devices, procedures and/or services contained thereon may be associated with keywords or other advertisements (e.g., via Internet search engines or other online locations) that, when clicked by a user, attract patients to that medical provider's profile upon the system. As mentioned above, a pay-per-click or click-through fee may be associated with such keywords or other advertisements, such that the medical provider corresponding to the profile pays die click-through fee to the owner of the system upon a successful linking of the potential user or patient to the profile. Thus, the corresponding medical provider may only pay die owner of the system upon successful attraction of a potential customer, rather than having to pay upfront for advertisements that might not bring any customers to the profile.

The owner of the system and/or the healthcare provider may be responsible for determining where such keywords or other advertisements are placed. For example, potential areas might be one or more search engines, advertising networks, television, radio, print ads, etc. Only upon successful click-through or linking of a customer to the profile due to the advertisements would necessitate payment by the healthcare provider. Traditionally, healthcare advertising to attract patients is done by paying monthly subscription fees, a monthly fee to advertisers to create online advertisements, with upfront costs associated with no guarantees of performance. These traditional methods may entail risk since a healthcare provider may or may not receive a patient due to the advertising despite the outlay of expenses. Using the above-described advertising method, a healthcare provider may only pay the owner of the system if a user dicks on an advertisement. In certain embodiments, the healthcare provider may only pay the owner of the system if a user clicks on an advertisement and also subsequently schedules an appointment for a medical device, procedure and/or service.

The owner of the system maintaining the healthcare provider profile may be in charge of managing the keywords and/or advertisements of the healthcare provider profiles. For example, upon choosing to create a profile, the system may automatically determine keywords based upon the devices, procedures, and/or services that arc chosen by the healthcare provider to be offered or available as part of the profile. In another embodiment, the keywords may not automatically be generated by the system, but may be determined or selected by the owner of the system (or an employee) such that consistent keywords are used across all healthcare provider profiles. Moreover, in certain embodiments, the system or owner of the system (or an employee) may write content for advertisements, set advertising budgets, and/or determine bidding on terms for advertisements, etc. in order to help save the costs seen by the healthcare provider.

For example, when creating or establishing a profile on the system, a healthcare provider may see a list of keywords that have already been selected by or in the system for advertising purposes and associate their devices, procedures, and/or services with those predefined keywords. In this fashion, by creating the profile, the healthcare provider need not worry about the additional tasks or expenses commonly associated with advertising since it will be handled by the system and/or the owner of the system. In an alternative embodiment, as mentioned above, advertising may be handled in manners in replacement of or in addition to, click-through based advertisement.

These procedures and services listed on the healthcare provider profile may be the same as those discussed herein that are available for potential customer selection and booking. In this manner, in addition or alternatively to the results shown to the user based upon user input previously discussed, a user may also be permitted to visit the profile of a desired healthcare provider (e.g., if a user inputs a specific hospital or geographic region, the results shown to the user by the system may include links to the profile page for such corresponding facilities and include procedures, services, etc. and the costs associated therewith).

In one embodiment, the viewer of the display 300 or user of the system is not charged any fee for use of the system. Alternative embodiments can utilize subscription plans or other monetary compensation from the users.

In some embodiments, if a user is satisfied with the costs displayed in step 292, the user can finish (i.e. stop interactions with the system). In this instance, the user can choose to accept costs based on one or medical providers, and can select costs based on at least a portion of the cost being provided by medical insurance and/or at least a portion of the cost being paid by cash by the user. In some instances, the user can select to pay at least a portion of foe cost even though that portion may be covered by a medical insurance policy. In other instances, the user can select to pay at least a portion of the cost that may not be covered by a medical insurance policy. In other embodiments, the user can select to pay all of the costs that are displayed in step 290.

In some farther embodiments, the user can proceed to review prior evaluations and/or book an appointment. For example, in some embodiments, after step 292b, the system can proceed with the steps and methods shown in FIG. 2A, including, but not limited to, steps 240, 245, 250, 255, and/or steps 260, 265.

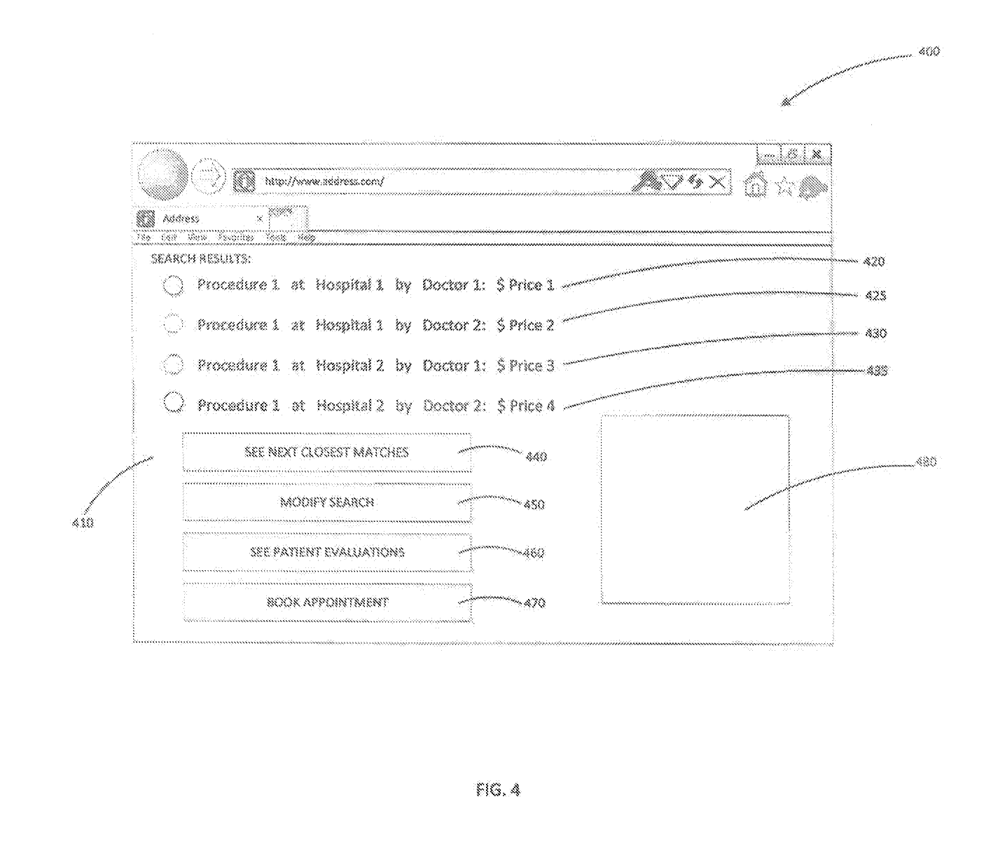

In some embodiments, a user can further review, evaluate, and select one or more procedures or services. For example, FIG. 4 shows a display 400 to a user with results of a query, the display 400 a part of a system implementing a price transparency medical procedure search. The system can Include features that are the same as or similar to those previously discussed, For example, the display 400 can be part of a system that allows a user to search for information regarding a desired medical procedure or service, such as previously discussed for FIGS. 1, 2A-2B, and/or 3. As illustrated, the display 400 can be a webpage that is accessed using a standard browser (e.g., Internet Explorer®, Google Chrome®, Mozilla Firefox®, etc.) and includes content 410 that is displayed in the browser,

The display 400 may include a plurality of search results (420, 425, 430, 435), These search results (420, 425, 430, 435) can be the result of a processor receiving user input data from a user or viewer of the system implementing the display 400 and using the data to search in a database or other memory for obtaining price or cost information, for example as discussed above in FIGS. 1, 2A-2B, and/or 3. For example, as illustrated, if a user of the system indicated a desired medical procedure (e.g., “Procedure 1” and indicated a desired location (e.g., location encompassing “Hospital 1” and “Hospital 2” within Its borders, with “Doctor 1” and “Doctor 2” in such area and available to perform particular medical procedure), those four search results (420, 425, 430, 435) and their associated costs (e.g., “Price 1,” “Price 2,” “Price 3,” and “Price 4,” respectively) can be shown. In some embodiments, if additional search results exist based on the desired medical procedure and location, the user of the system can click a button 440 to receive additional results. In an alternative embodiment, greater or fewer numbers of search results can be displayed to a user in any of a variety of possible formats (e.g., displayed one per page, displayed per location, etc.).

In some embodiments, if the user wishes to modify the search in some fashion, the button 450 can be pressed. This can present a query screen (e.g., display 300 of FIG. 3) to the user. If the user wishes to see patient evaluations, for example, user evaluations, reviews, data, qualifications, and/or statistics, the same as or similar to those previously discussed, button 460 can be pressed. In some embodiments, the search results (420, 425, 430, 435) can be presented as radio buttons such that the user can click to select one or more of the search results (420, 425, 430, 435) and then press the button 460 to get more detailed information that is relevant to the selected search result. Likewise, if a particular search result is desirable to a user, the user can click to select such search result and then press a button 470 allowing them to hook or schedule an appointment for the medical procedure. Similar to FIG. 3, advertisements 480 can be provided on the display 400 for providing a source of revenue for the system implementing the display 400.

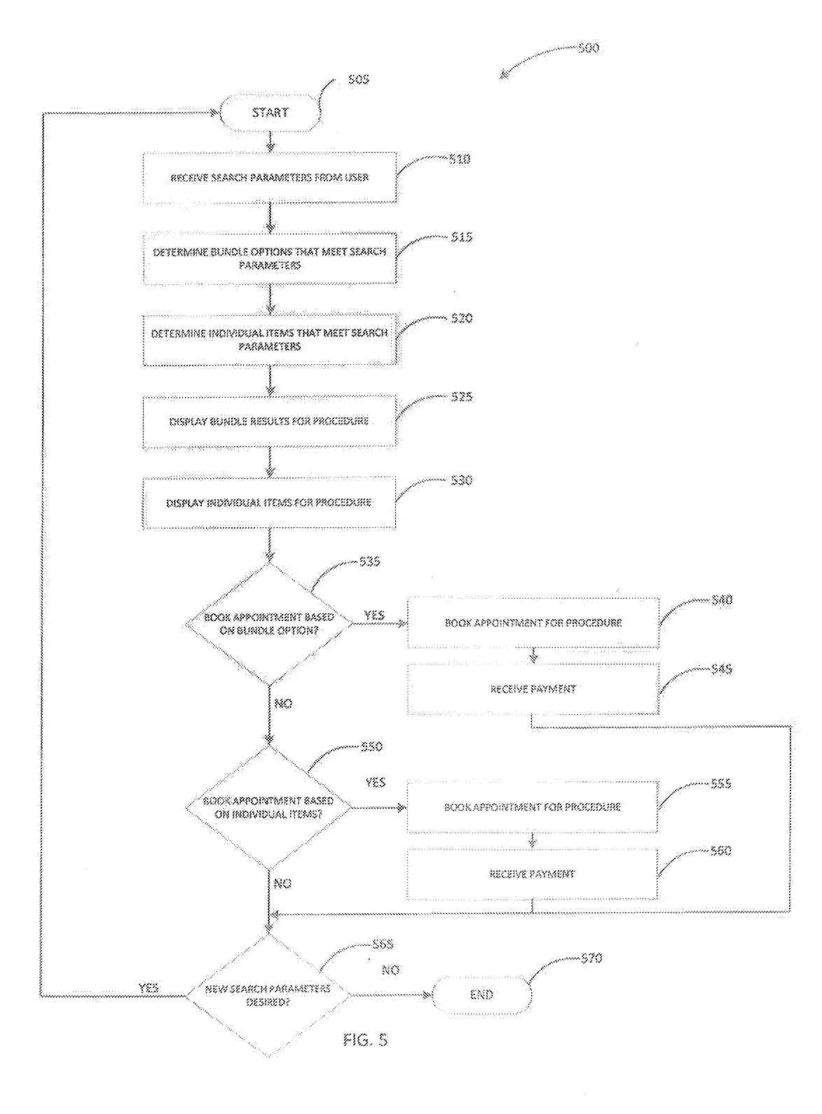

Turning next to FIG. 5, a flowchart of a process or method 500 for implementing a price transparency medical procedure search with bundling is shown. The process or method can include features that are the same as or similar to those previously discussed, and can comprise information derived from any of the systems and methods embodied by the steps shown in FIGS. 2A and 2B, or a combination thereof. In some embodiments, the bundled costs can comprise costs associated with one or more providers, including in-network and/or out-of-network providers. In other embodiments, the bundled costs can include one or more bundled cash-only options. For example, in reference to the example embodiments illustrated in FIG. 2B, following completion of step 280 or step 278, the processor can determine cash-only costs based at least in part on the healthcare provider information from at least one of the steps 274, 276, 278, and/or 280, In some embodiments, the processor can determine cash-only costs from any In-network providers (shown as step 282a), and/or for any out-of-network providers (shown as step 282b). In some embodiments, a user can compare total costs for cash-only payment costs, and can view a cash payment cost that can include instances where the patient pays up to 100% of the cost. Using these methods, a user can be provided with the option to view cash-only bundled cost before receiving a medical procedures or service,

In FIG. 5, a selection of predetermined bundle packages can be determined for the user and displayed to the user for selection, as discussed in greater detail herein. The process or method 500 can be implemented in a system that utilizes a processor for evaluating software instructions or code and a memory interfacing with the processor (e.g., as previously discussed for FIG. 1). A system the same as or similar to those previously described can be used in one embodiment. Thus, in at least one embodiments, a webpage can be established that allows a user to interact with various interactive elements or controls (e.g., drop-down boxes, checkboxes, radio buttons, text boxes, buttons, etc.) in order to determine a desired medical procedure for the user, as discussed in greater detail below. Moreover, a user determines and/or receiving costs for at least one medical procedure or service can receive the cost information through an internet service and/or internet web portal. In some embodiments, the internet service and/or internet web portal can provide an interface including tools for providing the cost comparison. More specifically, in some embodiments, the cost comparison or cost comparison tool can be accessed through single internet service and/or internet web portal without the need to switch between multiple internet services and/or internet web portals. In some embodiments, the cost comparison displayed through the internet service and/or internet web portal can include an insured component, a non-insured component, and/or a cash-only payment option component.

At step 505, the process starts. In some embodiments, this can occur when a user clicks on a link to arrive at or otherwise enters a webpage URL into an Internet browser on their personal computer, tablet, smartphone, etc. At step 510, the processor (e.g., processor 105 as discussed previously for FIG. 1) can receive search parameters from the user. Tills can occur by allowing the user to type, click, or otherwise select among different criteria (e.g., medical procedures, surgeries, medical services, date/time, geographic location, hospital, doctor, etc.) in order to determine the interests of the user. This can be determined by examining input from the user that is received via one or more interactive elements or controls placed on a webpage using the system and methods embodied by either of FIGS. 2A and 2B as described earlier. For example, a dropdown box or a textbox can be disposed on the webpage and ask for the user to indicate, by selecting or typing, respectively, the geographic area where they would like to have a medical procedure performed. The interactive elements or controls can be located upon a single webpage screen or can span a plurality of webpage screens which a user clicks through and makes selections thereon.

The same as or similar to tire previous discussions, the interactive elements can function by asking the user to indicate a city, state, zip code, address, or the like and can also indicate a desired radius (for example, in miles) that represents the maximum geographic distance from the indicated address that the user would be willing to travel for performance of the medical procedure. In some embodiments, a map can be displayed on the webpage to allow the user to graphically pinpoint the address and desired radius upon the graphical map. Certain embodiments can allow a user to search in a variety of different countries (e.g., United States, Europe, Canada, etc.) or worldwide.

In some embodiments, at step 515, the processor can determine one or more bundle options or packages that meet one or more of the search parameters received in step 510. The bundle options or packages can represent groups or collections of medical procedures/services and/or other types of services or items that are combined together into one selectable package by the user. A bundle cost or price for each of the bundle options or packages can also be determined in some embodiments. In at least one embodiment, the bundle cost is a lower cost than if the user had opted to individually select each of the medical procedures/services and/or other types of services or items that, are combined together to form the bundle. For example, this lower cost can be obtained by having predetermined contracts or agreements with particular vendors of goods or services, where they agree to offer such goods or services at a lower price in order to be included in the bundle packaging, in other words, in some embodiments, the particular vendors of goods or services can comprise in-network providers as discussed earlier. In one embodiment, only bundle options or packages that meet all of the user-desired search parameters in step 510 are determined for selection. In an alternative embodiment, bundle packages can be determined for selection by the user if they meet only some of the search parameters in step 510, but not all of them. In some embodiments, the cost comparison displayed can include an insured component. In other embodiments, the cost comparison can exclude an insured component. For example, the cost component can comprise a cash-only comparison of bundled packages.

At step 520, the processor can determine individual items that can be selected by the user for purchase that meet one or more of the search parameters received in step Sid. A corresponding cost for each of the individual Items may also be determined. Similar to previous discussions, price data for a variety of medical procedures, services, and/or ancillary goods or services can be stored in a database or otherwise obtainable by connection to a remote system or server and obtained when the search parameters received in step 510 indicates such medical procedures, services, and/or ancillary goods or services meet the user's desires,

At step 525, the results of the determination made by the processor in step 515 can be displayed to the user. In some embodiments, the display of the results can be a single bundle package with a corresponding bundle cost that was determined to meet the user's search parameters from step 510, or can be a list of bundle packages with corresponding bundle prices. In this manner, the user can browse the list for deciding the bundle package according to their preferences. The results can all be displayed at once to the user or can be displayed across various screens or tabs that require the user to click or otherwise manipulate a control on a webpage to see additional results. The results can be organized for display according to various schemes (e.g., from lowest price to highest price, from closest geographic location to furthest geographic location, etc.) Further, in some embodiments of the invention, a user can provide updated information (e.g., in steps 274 and/or 276 as previously discussed for FIGS. 2A and/or 2B) based on preferences for one or more in-network or out-of-network providers, copayment cost, deductible cost, cash-only cost, and other expenses associated with the medical procedure or service. In some embodiments, a user can attempt to modify costs displayed by updating input provided to either or both of steps 274 and/or 276. For example, in some embodiments, preferences for in-network or out-of-network provider's copayment cost, deductible cost, cash-only cost and other expenses associated with the medical procedure or service can be provided or updated. In certain embodiments, the user can manipulate how the results are organized for display.

Similarly, at step 530, the results of the determination made by the processor in step 520 are displayed to the user. The display of the results includes each of the individual procedures, services, and/or items and a corresponding cost or price for each individual procedure, service, and/or item that were determined in step 520 to match the user's search parameters in step 510. In this manner, if the user would rather choose specific options, rather than the, perhaps more limited, options available in the bundle packages, the user is free to select according to those precise desires. For example, while the bundle results displayed in step 525 can only correspond to particular hospitals, doctors, etc. that have agreed to offer their goods or services for a reduced cost as part of bundle deals, if the user desires a specific hospital or doctor not among the bundled options, the user can choose according to those preferences via the procedures, services, and/or items.

In some embodiments, the results displayed to the user can comprise bundled costs calculated by the processor based on the patient's copay and/or the patient's deductible. For example, in some embodiments, the bundled costs can be adjusted based on a patient's copayment responsibility. Further, in some embodiments, the bundled costs displayed can be adjusted based on a patient's deductible responsibility. In some embodiments, the bundled costs displayed can be adjusted based on a patient's copayment and deductible responsibility. In some embodiments, any adjustments to the bandied cost can be based on the patient's prior payment history. For example, in some embodiments, an adjustment can be made based on the patient's remaining deductible that comprises an adjusted deductible based on at least one previous medical procedure or service. Further, in some embodiments, the bundled costs can comprise a comparison of cash-only payment costs compared with out-of-pocket expenses that can comprise copayment costs and/or deductible costs.

In at least one embodiment, the cost to the user for making individual selections can be greater than if the same selections were chosen as part of a bundled package. The same as or similar to the previous discussion, the results can all be displayed at once to the user or can be displayed across various screens or tabs that require the user to click or otherwise manipulate a control on a webpage to see additional results. The results can be organized for display according to various schemes (e.g., from lowest price to highest price, from closest geographic location to furthest geographic location, etc.). In certain embodiments, the user can manipulate how the results are organized for display.

At step 535, it can be determined whether the user wishes to book an appointment for one of the bundle package options displayed in step 525. This can occur, for example, in response to the user selecting one of the bundle options displayed and clicking on a link indicating a desire to schedule an appointment per the selected result. In some embodiments, the user can be presented with an option to book an appointment for one of the bundle package options displayed m step 525 that comprise an insurance-paid cost portion, in some other embodiments, the user can book an appointment for one of the bundle package options displayed in step 525 that comprises a cash-only portion. In some embodiments, the cash-only option comprises 100% of the total cost of services to be rendered at the appointment. If an appointment is desired, operation continues to step 540, If no appointment is desired, operation continues to step 550, discussed in greater detail below.

At step 540, available times and/or dates for the medical procedure at the selected result can be displayed for the user to browse and choose a desired appointment time. In some embodiments, the user can be able to select a desired span of dates or times and the processor will determine and/or cause to display available appointment times within that span. In other embodiments, the bundle package can be only for a predetermined date. At step 545, the processor can receive payment information (e.g., a credit card number, a PayPal® account, a deposit account number, etc.) from the user to pay for all or a portion of the bundle package option. In some embodiments, no payment may be required. Operation may then continue to step 565, as discussed in greater detail below,

At step 550, it can be determined whether the user wishes to book an appointment for one or more individual items displayed in step 530. This can occur, for example, in response to the user selecting one or more of the individual items displayed and clicking on a link Indicating a desire to schedule an appointment or to purchase the selected result. If an appointment or purchase is desired, operation continues to step 555. If no appointment or purchase is desired, operation continues to step 565, discussed in greater detail below. At step 555, available times and/or dates, if applicable, or other purchase input information as desired for the one or more individual items can be displayed for the user to browse and provide additional information as needed. At step 560, similar to the discussion above, the processor can receive payment information (e.g., a credit card number, a PayPal® account, a deposit account number, etc.) from the user to pay for all or a portion of the individual items that were selected. In some embodiments, no payment may be required.

In some embodiments of the invention, operation then continues to step 565 to determine whether new (e.g., modified) search parameters are desired by the user. This can occur, for example, in response to user input indicating they wish to modify a current or already existing search that they have performed or whether they wish to begin a new search from the beginning. If no new search parameters are desired, operation continues to step 570 where the process ends. However, if new search parameters are desired, operation continues back to step 505 where the process starts anew. Alternative embodiments can utilize additional or fewer steps than those explicitly shown in the exemplary flowchart of FIG. 5. Certain steps can be combined. Certain steps can be omitted. Additional steps may be inserted. Steps can additionally or alternative be performed in a differing order from those explicitly shown and described herein.

Any of a variety of search parameters can be used for generating search results for a user, either for bundle package options and/or for individual item options. For example, in some embodiments, a user can desire to browse bundle package options that are available that include a particular doctor, surgeon, or other medical practitioner at a particular hospital or other medical facility. Further, in some embodiments, any bundle option can include preferences for in-network and out-of-network providers, and selections based at least in part on the patient's copayment responsibility, the patient's deductible, ancillary expenses, cash-only options, and/or other expenses associated with the medical procedure or service (such as those determined by the system and methods illustrated by the flowchart 270 shown in FIG. 2B).

In this manner, the user can obtain an inexpensive and comprehensive price by browsing and selecting a bundle package option, but still maintain control over the medical personnel and/or facilities performing or involved in the procedure. If a user is Instead more interested in price and/or location, and less interested in the medical practitioner and/or facility, those alternative search parameters can be entered by the user and used for determining the search results. By allowing the user flexibility to enter as many or as few search parameters as they desire, more customized search results can be obtained for the user to select amongst.

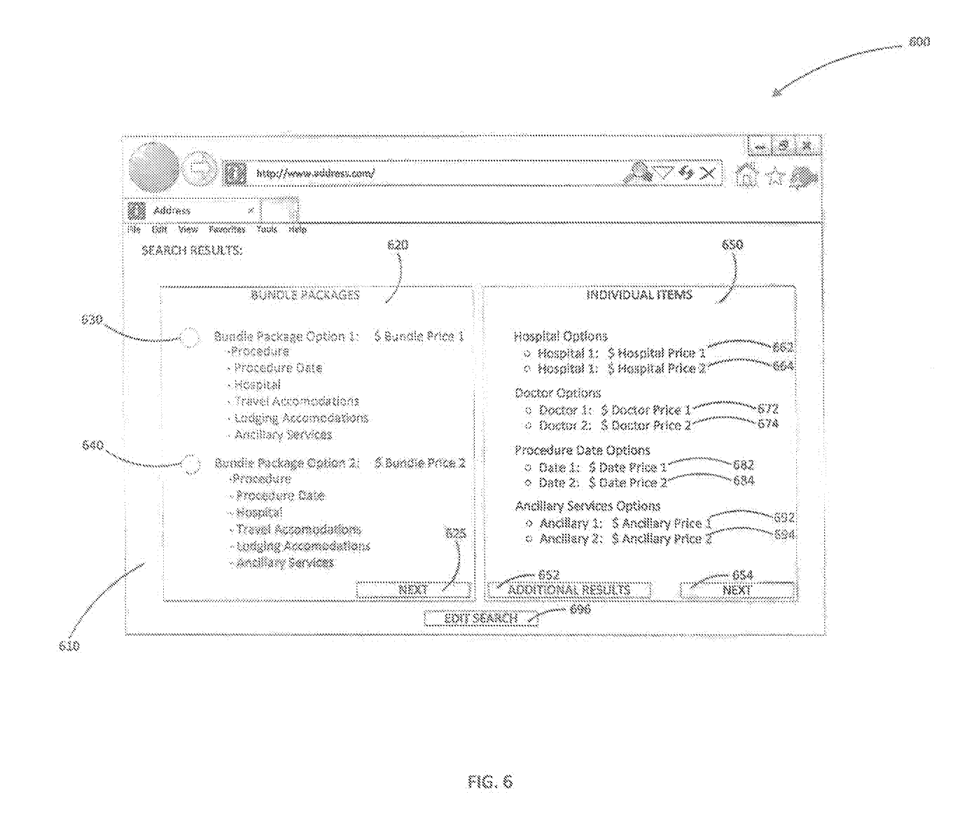

FIG. 6 shows a display 600 to a user with results of a query, the display 600 a part of a system implementing a price transparency medical procedure search with bundling. In some embodiments, the system can include features that are the same as or similar to those previously discussed. For example, the display 600 can be part of a system that allows a user to search for information regarding a desired medical procedure, such as previously discussed in relation to the system and methods depicted in FIGS. 2A, 2BS and/or 3. As illustrated, the display 600 can be a webpage that is accessed using a standard browser (e.g., Internet Explorer®, Google Chrome®, Mozilla Firefox®, etc.) and includes content 610 that is displayed in the browser.

In some embodiments of the invention, the system can use search parameters received from the user in order to determine bundle options and/or individual items to offer for sale to the user, the same as or similar to the previous discussions. After making such determinations, the system showcases such results to the user upon a display screen where the user is able to make selections according to their preferences. The display 600, according to some embodiments as illustrated in FIG. 6, includes bundle package options 620 and individual (e.g., a la carte) item options 650 that can be selected by the user for purchase in some embodiments of the invention. Regarding the bundle package options 620, a first bundle package option 630 and a second bundle package option 640 are shown and may be selectable by the user (e.g., by clicking on a radio button, checkbox, or other interactive dement that is displayed on die display 600). In an alternative embodiment, greater or fewer bundle package options eats be displayed to the user for selection.

The first bundle package option 630 has a corresponding bundle cost or price that represents the cost to the user for all of the goods or services provided as part of the first bundle package option 630. In some embodiments, these costs can be calculated by a processor (e.g., processor 105 of FIG. 1) using any one or combination of embodiments shown in FIGS. 2A-2B, and/or 3. For example, these goods or services can include some or all of the following: a medical procedure to be performed, the date and/or time of the performance of the medical procedure, hospital costs (e.g., room and board, meals, etc.) associated with the performance of the medical procedure, travel accommodations (e.g., airfare, bus fare, train fees, taxi costs, shuttle costs, car rentals, etc.) in order to locate the user desiring the medical procedure from their residence to the hospital, lodging accommodations (e.g., hotel costs, after-care facilities, etc.) if the user will be away from their residence before and/or after the medical procedure is performed, and other, ancillary' services (e.g., after-care treatment such as rehabilitation, etc.).

In some embodiments, any of the costs can comprise costs based on a user selection during any of the systems and methods depicted in FIGS. 2A-2B, and/or 3. The costs may comprise costs derived following a user selection of in-network provider, out-of-network provider, range or maximum of copay, range or maximum of deductible, range or maximum or other expenses, cash-only options, or selection or de-selection of any ancillary services. Moreover, the costs may comprise bundled costs calculated by the processor based on the patient's copay and/or the patient's deductible. For example, the bundled costs can be adjusted based on a patient's copayment responsibility. Alternatively, or additionally, the bundled costs displayed can be adjusted based on a patient's deductible responsibility. In some embodiments, the bundled costs displayed can be adjusted based on a patient's copayment and deductible responsibility.

Adjustments to the bundled cost may be based on the patient's prior payment history. For example, an adjustment can be made based on the patient's remaining deductible that comprises an adjusted deductible based on at least one previous medical procedure or service. Further, in some embodiments, the bundled costs displayed can comprise a comparison of cash-only payment costs compared with out-of-pocket expenses that can comprise copayment costs and/or deductible costs.

Similarly, the second bundle package option 640 has a corresponding bundle cost or price that represents the cost to the user for all of the goods or services provided as part of the second bundle package option 640. The goods or services offered as part of the second bundle package option 640 can be the same as or similar to those offered by the first bundle package option 630, as discussed above. For example, the same goods or services can be offered in both of the first bundle package option 630 and the second bundle package option 640, but at different hospital locations and/or at different dates and/or times. Such differences can result in different associated costs or prices for each of the first bundle package option 630 and the second bundle package option 640, In certain embodiments, the costs associated with the first bundle package option 630 and the second bundle package option 640 can be the same,

By comparing the costs and/or the goods or services offered by the first bundle package option 630 and the second bundle package option 640, the user can decide which bundle is more desirable and opt to select one of the bundles in order to book an appointment for the medical procedure and/or purchase the goods or services being offered. As previously discussed, the system implementing a price transparency medical procedure search with bundling can have contracts, agreements, or other arrangements with various vendors and/or providers of goods or services that have agreed to participate in bundling of their goods or services in exchange for offering such goods or services at a reduced price. For example, a vendor or provider can feel the increased quantity of goods or services sold as a result of bundled participation warrants a reduction in price in order to obtain those sales. In one embodiment and as shown, upon selecting the first bundle package option 630 or the second bundle, package option 640, the user can click or select a next button 625 that continues system operation to a booking, reservation, or other purchase setup, for example, as previously discussed.

In order to accommodate users who do not wish to purchase bundle packages (e.g., users with particular preferences for doctors, hospitals, travel arrangements, etc.) the system may also or alternatively allow users to specifically chose individual items that represent goods and/or services based upon the search parameters received from the user. For example, if the user indicated via the search parameters that they currently reside in California and desire Lasik surgery, bundle options can be shown for particular hospitals and/or doctors in the nearby geographic area that have agreed to participate in bundling of corresponding goods and/or services, but the user may have a particular hospital or doctor that they wish to see or may not need or may desire to make their own travel accommodations. Rather than only being able to choose a bundled option, such a user can instead more specifically choose the individual components of or relating to the desired procedure.

In some embodiments of the invention, the display 600 can show to the user the individual item options 650 that can be selected by the user for purchase. The same as or similar to previous discussions, these individual item options 650 can be based upon search parameters previously received from the user, and can include insured costs, uninsured costs, and cash-only payment options. For example, a first hospital option 662 with a corresponding first hospital price, a second hospital option 664 with a corresponding second hospital price, a first doctor option 672 with a corresponding first doctor price, and a second doctor option 674 with a corresponding second doctor price can be shown. In some embodiments, the user can then specifically choose the hospital and the doctor for the procedure,

In addition, a first date option 682 with a corresponding first date price and a second date option 684 with a corresponding second date price can be shown. The user is thus permitted to choose a specific date and/or time for the desired procedure to be performed. In some embodiments, at least some of the procedure can include an insured cost amount, in other embodiments, the desired procedure can be a cash-only option selected by the user, [00122] A first ancillary option 692 with a corresponding first ancillary price and a second ancillary option 694 with a corresponding second ancillary price may also be displayed, allowing users to choose between and/or to choose more than one ancillary service that is related to the desired procedure (e.g., aftercare services, medications purchases, medical equipment purchases, etc.). In some embodiments, one or more of the individual item options 650 are selectable by the user (e.g., by clicking on a radio button, checkbox, or other interactive element that is displayed on the display 600). in an alternative embodiment, greater or fewer individual item options can be displayed to the user for selection.

By comparing the costs and/or the goods or services offered fey the various individual item options 650, the user can decide which items are desirable and opt to select one or more of the individual items for purchase. For example, in the example above, in some embodiments, at least a portion of any ancillary price can be selected by a user to be designated as at least partially paid for by any available medical insurance. In other embodiments, the user can select to pay for any ancillary services using a cash-only option.

In some embodiments, and as show, upon selecting one or more of the individual item options 650, the user can click or select a next button 654 that continues system operation to a booking, reservation, or other purchase setup, for example as previously discussed. If more individual items are available and offered to the user than can be displayed at once on the display 600, the user can click or select an additional results button 652 in order to view a next page or listing of individual item options 650 that can be purchased. If the user decides that either or both of the bundle packages 620 and/or the individual item options 650 do not correspond to their desires, an edit search 696 button can foe clicked or selected in order to re-enter or modify some or all of the search parameters.

In addition to, or alternatively to, the various features described fey the specific embodiments above, financing features may also be provided to users seeking healthcare procedures or services. For example, as discussed in greater detail herein, one or more financing options from a banking or lending institution may be provided for user selection or confirmation if the user desires to finance some or all of die costs associated with their healthcare procedure or service. Banking or lending Institutions may partner with, or otherwise cooperate or interface with a system that provides users with healthcare searching capabilities (or, in an alternative embodiment, as a standalone system just providing financing capabilities) the ability to browse, search, and/or select desired financing possibilities.

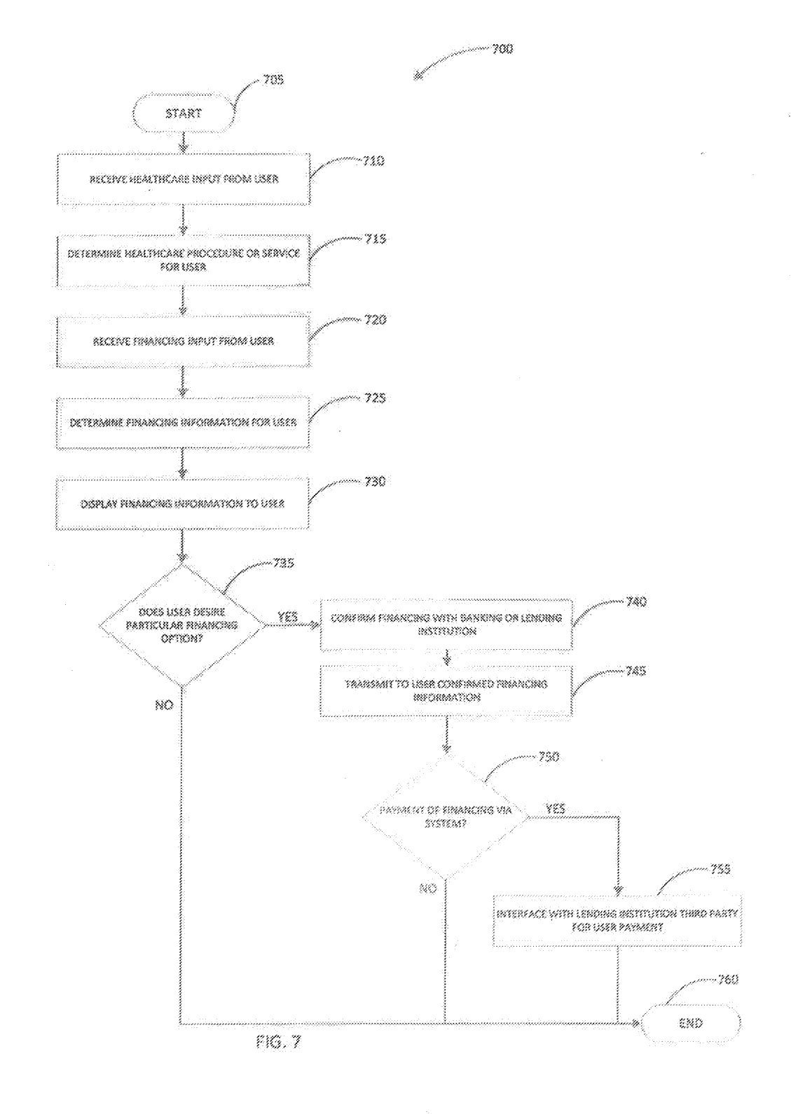

For example, FIG. 7 illustrates a flowchart of a process or method 700 for implementing one or more financing options in a healthcare procedure or services solution. In some embodiments, the same or similar to previous discussions, the process 700 can be implemented in a system that utilizes a processor for evaluating software instructions or code and a memory interfacing with the processor. For example, a system the same as or similar to that described above and shown in FIG. 1 or elsewhere can be used In at least one embodiment of the invention (e.g., utilizing processor 105 and/or memory 110). Thus, in at least one embodiment, a webpage or other software application (e.g., configured to be executed on a mobile device such as a smart phone, tablet, laptop, etc.) can be established that allows a user to interact with various interactive elements or controls (e.g., drop-down boxes, checkboxes, radio buttons, text boxes, buttons, etc.) in order to search and/or select desired financing options, as discussed in greater detail below.

At step 705, the process 700 starts. In one example embodiment, this can occur when a user clicks on a link to arrive at or otherwise enters a webpage URL into an Internet or network browser on their personal computer, tablet, smartphone, etc. Other embodiments can utilize alternative ways for allowing user interaction besides a webpage (e.g., a computer software executable, such as a smartphone or tablet application, etc.). In certain embodiments, the process 700 may occur subsequent to other processes of a webpage or software application (e.g., a user may first choose one or more healthcare procedures or services, for example, as described previously, and then continue to a financing options process for such desired healthcare procedures or services), as discussed in greater detail herein.

At step 710, the system receives healthcare input from the user. As previously mentioned, this may include input from a user that is the same as or similar to previous discussions regarding a desired medical or other healthcare procedure, service, bundle, etc. that the user would like to have performed. At step 715, the system determines (e.g., with additional user feedback or not, in certain embodiments), the desired healthcare procedure or service for the user and/or other related characteristics (e.g., specific doctor, specific hospital, specific time, specific price, etc.). At step 720, the system receives financing input from the user. In one embodiment, such financing input may merely be input that indicates the user desires to begin a financing options process via the system rather than funding the desired healthcare procedure or service by other means.

In some embodiments, any of a variety of alternative and/or additional inputs may be asked for and/or received from the user as financing input. For example, the user may be able to specify a desired or preferred banking or lending institution, a desired or preferred financing rate (e.g., may set a maximum rate that the user would consider for any financing option), a desired or preferred financing time period (e.g., may set a maximum or a minimum length of time or number of payments that the user would consider for any financing option), a desired or preferred amount for financing (e.g., the entire purchase price for the healthcare procedure or service, or a greater or lesser amount than the entire purchase price), etc. Alternative embodiments may utilize any number of financing data and/or inputs from a user in helping determine financing information for communication to the user,

At step 725, the system determines financing information for the user, for example, based upon one or more of the financing input received in step 720. This financing information may include banking or lending institutions that will or may offer the user financing, particular finance rates associated with a loan, number of payments associated with a loan, or any of a variety of other information that may be pertinent or desirable by a user to have access to in making a decision on whether to accept such a financing agreement. At step 730, some or all of this financing information may be displayed to the user. This display may be via a display screen or may be via a communication or transmission to the user that the user can display or otherwise interpret in other software or means (e.g., an email, text message, snail mail, etc.). In some embodiments, only one loan (with any desired associated information) option may be displayed to a user. In alternative embodiments, a plurality of loan options may be displayed such that the user can choose the one that is most fitting according to their preferences.

At step 735, the user is allowed to select or otherwise indicate a desire for a particular financing option, based upon the display of financing information, for example, as described in step 730, If the user indicates such a desire (e.g., by checking a checkbox, button, or other user interface element, that corresponds to one of the financing options), the process 700 continues to step 740. At step 740, the system confirms with the requisite banking or lending institution that such a financing option will be available for the user. For example, this may include the transmittal of one or more additional information to the banking or lending institution. In some embodiments, this additional information may include information about the user such that the banking or lending institution may perform a credit check or other verification about the user's finances or ability to pay back the loan. If this confirmation is not satisfied (e.g., the additional information about die user indicates an unacceptable risk to the banking or lending institution), the banking or lending institution may send a communication back to the system denying the financing possibility and/or providing alternative terms (e.g., a greater down payment, a greater loan rate, etc.) that the user may choose to accept or decline.

Once an appropriate confirmation by the banking or lending institution occurs, however, at step 740, such a final confirmation is transmitted to the user with the final details of the financing. This may be in the form of a summary screen transmitted from the banking or lending institution and subsequently displayed to the user. In certain embodiments, the user may be required to perform a final authorization (e.g., clicking of an “Accept” button or user-interface dement) in order to confirm and agree to be hound by the final terms of the financing arrangement with the banking or lending institution. In other embodiments, no further confirmation may be required, and the user's prior selection of the financing details (e.g., as described at step 735) may be deemed the user's acceptance of the terms. In still other embodiments, a final confirmation transmitted to the user may be performed outside of the bounds of the system itself (e.g., a snail mail, email message, or other communication may be sent directly to the user, for example, to receive signature).

In certain embodiments, the system may also be configured to manage and/or control payments made as part of the financing arrangement entered into via the system. At step 750, the system may request feedback from the user as to whether the user desires to may payments electronically via the system to the banking or lending institution. If so, operation continues to step 755, whereby the system provides a payment interface that communicates with the banking or lending institution (or a third party that operates in conjunction with the banking or lending institution) to accept user payment, provide billing information to the user, etc. Any of a variety of possible payment methods may be utilized by such payment Interface (e.g., credit card payments, electronic transfers, wire transfers, banking account transfers, etc.). At step 760, the process ends. If the user did opt to make one or more payments via the system per step 750, the user may be able to log in to the system at subsequent times and immediately choose to go to the payment interface for viewing data or information about their current, past, or pending potential future financing options and/or making payments. Returning to step 750, if the user opts not to make payments as part of the system (e.g., the user prefers to mail checks or otherwise control payments outside of the system, the process continues immediately to step 760, bypassing the payment interface. Similarly, returning to step 735, if the user indicates that there are no desirable financing options available that they wish to sign up for or receive additional information about, operation also continues immediately to step 760.

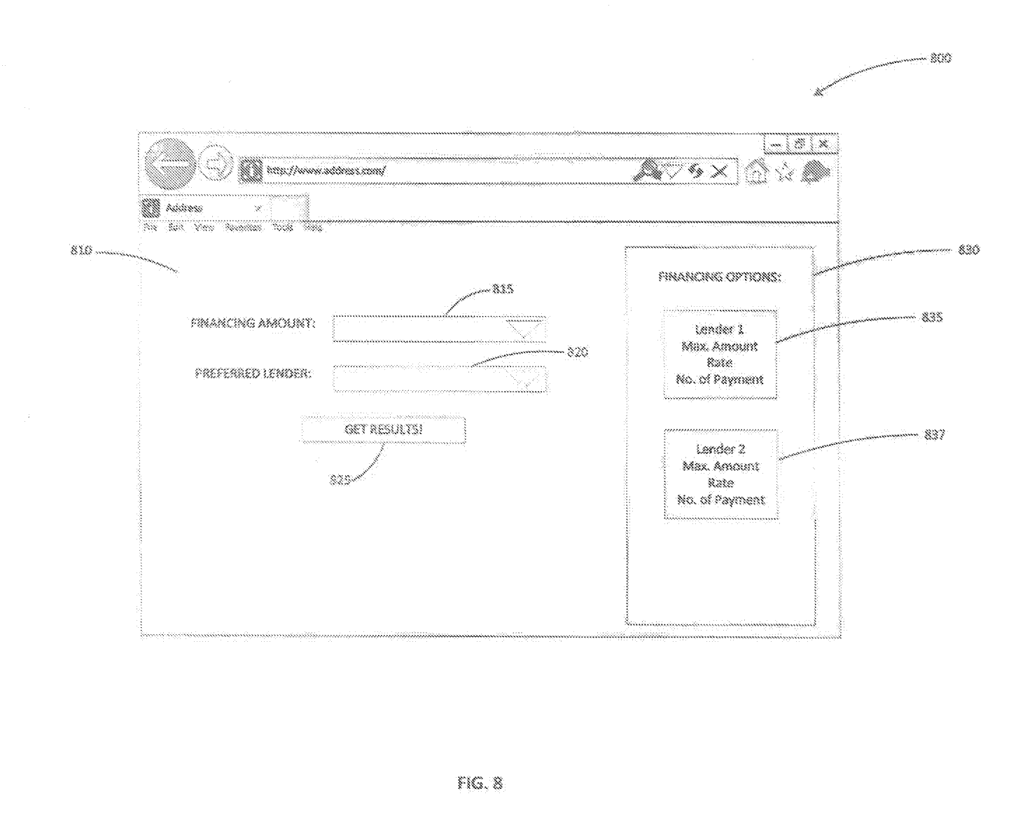

FIG. 8 Illustrates a display 800, such as a visual display screen, that may be provided to a user for allowing a user to provide financing preferences and/or for displaying results of a financing options query (that may or may not be based upon the financing preferences of the user), as discussed in greater detail below. Certain aspects or features of the display screen may be the same as or similar to previous discussions. For example, one or more of the steps previously described in FIG. 7 may be provided for by the display 800 of FIG. 8. Alternative embodiments may utilize displays (or other communication methods to a user) that differ in function and/or appearance than the embodiment explicitly illustrated in FIG. 8.

The display 800 may be a part of a system implementing a healthcare procedure and/or services solution that includes searching capabilities. As illustrated, some embodiments of the invention can include the display 800 that can comprise a webpage that Is accessed using a standard Internet browser (e.g., Internet Explorer®, Google Chrome®, Mozilla Firefox®, etc.) and includes content 810 that is displayed in the browser. Internet Explorer® is a registered trademark of the Microsoft Corporation in the United States and/other countries. Firefox® Is a registered trademark of the Mozilla Foundation, Google Chrome is a trademark of Google Inc. Alternative embodiments may include the display 800 as part of a software application that Is configured to be executed by a processor, such as a processor within a mobile device, like a smart phone.

A first user interface element SIS is displayed and corresponds to a desired financing amount. In certain embodiments, this financing amount may correspond to a purchase price or cost associated with a healthcare procedure or service (e.g., one that was previously searched for and/or selected by the user). The financing amount may be less than, greater than, or equal to the purchase price or cost. As shown, the user may be allowed to specify what the financing amount will be that is desired. For example, if the cost of a particular medical procedure was $50k, but the user also wished to include in the financing additional money (e.g., money to cover expenses while the user is unable to work due to the procedure), the user can indicate the desired amount of financing by interfacing with the user interface element 815, Although a dropdown box is specifically illustrated in FIG. S, any of a variety of interface elements may be used in alternative embodiments (e.g., slider bars, text boxes, etc.)

Similarly, a second user interface element 820 is displayed and corresponds to a desired or preferred lender that the user may wish to use. For example, a user may have an account with a particular banking institution and/or may have additional or prior financing loans from other lending institutions that they wish to continue to use their services for future financing options. The user may interface with the user interface element 820 to choose or select such a preferred lender. Multiple preferred lenders may be chosen by a user in alternative embodiments. Similar to the discussion above, although a dropdown box is specifically illustrated for user interface element 820 in FIG. 8, any of a variety of interface dements may be used in alternative embodiments (e.g., text boxes, cheek boxes, radio buttons, etc.). Likewise, although only two specific user interface elements (815, 820) are shown in FIG. 8, alternative embodiments may have greater or fewer depending on the number of financing inputs desired to make available for user selection. A third user interface element 825 (e.g., illustrated as a clickable button) is displayed and allows a user to finalize or send their chosen financing inputs to the system so that the system may determine available financing options for the user.

Upon interfacing with the third user interface element 825, one or more results may appear in the results content 830. in certain embodiments, if nothing matches the preferences supplied by the user, the results content 830 may display “No results available” or a similar notification to indicate to the user that one or more of tire preferences should be adjusted in order to obtain results. Some embodiments may always display at least one result, even if it does not explicitly match every preference previously indicated by the user. As illustrated in the exemplary FIG. 8, a first option 835 with associated financing information (e.g., lender entity, max amount for financing, finance rate, number of payments, etc.) may be displayed. Likewise, a second option 837 with associated financing information (e.g., lender entity, max amount for financing, finance rate, number of payments, etc.) may be displayed. In alternative embodiments, greater or fewer options may be shown to the user. The same or similar to previous discussions, such as for FIG. 7, the user may then be permitted to select one (or more than one) of the displayed financing option results in order to receive additional information there about and/or to sign up or attempt to sign up for such financing. In alternative embodiments, selection of one or more of the financing results may provide the user with information on how to contact the selected banking or lending institutions directly to proceed further, rather than performing any subsequent financing approval steps within the system itself,

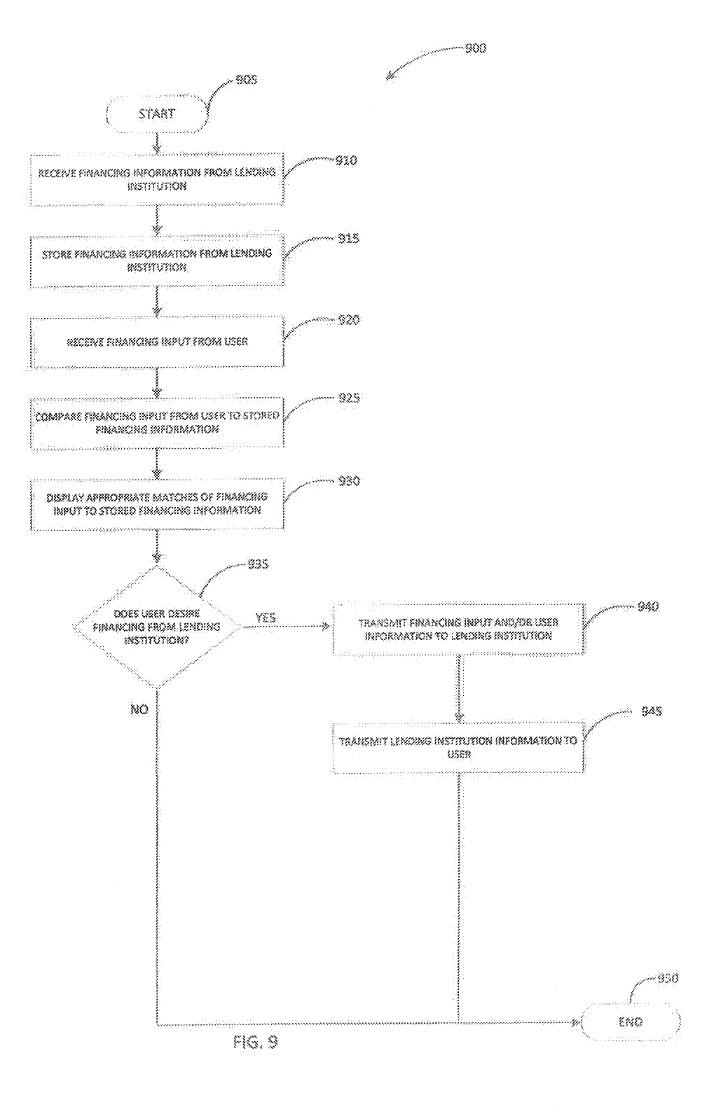

Various incentives may provide an impetus for one or more banking or lending institutions to become a part of or partner with a system that provides searching capabilities for the healthcare field, the same or similar to those previously discussed, in order to provide a platform for advertisement of their lending services, FIG. 9 illustrates an exemplary process or method 900 for implementing financing options in a healthcare procedure or services solution. In some embodiments, the same or similar to previous discussions, the process 900 can be implemented in a system that utilizes a processor for evaluating software instructions or code and a memory interfacing with the processor. For example, a system the same as or similar to that described above and shown in FIG. 1 or elsewhere can be used in at least one embodiment of the invention (e.g., utilizing processor 105 and/or memory 110), Thus, in at least one embodiment, a webpage or other software application (e.g., configured to be executed on a mobile device such as a smart phone, tablet, laptop, etc.) can be established that allows a user to interact with various interactive elements or controls (e.g., drop-down boxes, checkboxes, radio buttons, text boxes, buttons, etc.) in order to search and/or select desired financing options, as discussed in greater detail below.

At step 905, the process 900 starts, in one example embodiment, this can occur when a banking or lending institution agrees to sign up or partner with the system in order for its lending possibilities to be potentially made available for user viewing and/or selection and logs in or otherwise accesses the system. Accessing of the system may occur via entering a webpage URL into an Internet or network browser on their personal computer, tablet, smartphone, etc. Other embodiments can utilize alternative ways for allowing user interaction besides a webpage (e.g., a computer software executable, such as a smartphone or tablet application, etc.). At step 910, the system receives financing information from the banking or lending institution (e.g., name of institution, types of loans and/or procedures and/or services they are willing to cover, maximum amount of financing the institution is willing to offer, etc.). At step 915, one or more of this financing information may be stored by the system, for example, in a storage medium or memory feat is local to the system and/or otherwise accessible by fee system (e.g., via remote communication).

At step 920, the same or similar to previous discussions, one or more financing inputs may be received from a user (e.g., whether financing is desired, preferred amounts to finance, preferred Sending institutions, etc.). At step 925, one or more of the financing inputs received from fee user are compared against one or more of fee financing information stored in the system. In alternative embodiments, only some or no financial Information may be stored as part of the system, but rather, one or more financing inputs received from the user may be provided to the banking or lending institution and/or a third party operating on behalf or for fee banking or lending institution in order to determine appropriate financing options for the user based on the financing input. In still other embodiments, no financing input from the user may be needed (e.g., a list of available banking or lending institutions and/or associated financing information may be provided to the user, either from stored information of the system and/or information remote from fee system).

At step 930, based upon the determined or appropriate matches via the comparison of step 925, one or more financing options are displayed or otherwise communicated to the user. At step 935, the user indicates to the system whether they desire financing from a particular banking or lending institution, for example, using user interface elements of a display screen to select or choose one or more among the displayed financing options. If so, operation continues to step 940 where one or more of the financing inputs are transmitted to the particular banking or lending institution. Such operation may allow a preliminary set of financing options be displayed to the user, based upon basic, locally stored, financing possibilities, with further confirmation and/or specifics determined via communication directly to the banking or lending institution (e.g., user information and/or financing input that would allow the banking or lending institution to run a credit check or otherwise determine whether and under what terms financing may be provided to the user.). At step 945, the banking or lending institution transmits specific terms and/or approval of a particular financing agreement to the system and/or such specific terms and/or approval is communicated (e.g., displayed) to the user. At step 950, the process 900 ends. Likewise, at step 935 if the user indicated that there was not any desire for financing from the displayed options, operation would similarly continue to step 950.

Various financing features, such as those explicitly described for the specific embodiments illustrated, may be included as part of a larger system that includes searching functionality for healthcare procedures and/or services. In alternative embodiments, financing features may be separately embodied by a system without any other searching functionality. Various revenue may be obtained by owners and/or operators of such a system via the financing features. For example, a portion of the finance payments made by a user to a banking or lending institution may be provided to the owners and/or operators as a fee, banking or lending institutions may fee required to make one or more payments thereto in order to have their financing options browse-able, searchable, and/or displayed to users, advertisements related to financing options may be provided on one or more content portions of the system that can be viewed by users, etc.

Although the embodiments described above depict one or more systems with a variety of financing features, more simple systems may be provided that focus additionally and/or alternatively on connecting users with possible financing institutions, but without more intricate features. For example, in one embodiment, if a user indicates that they are interested in financing, the system may provide the user with contact information for particular financing institutions, may allow users to send communications via the system to particular financing institutions (e.g., via email or text message or video message), etc. Any of a variety of possible interfacing process and/or methods may be used for providing users with easier access to available financing options for medical procedures in alternative embodiments. As described throughout this specification, using one or more of the features described (e.g., searching, bundling, financing, etc.) in a system, process, and/or method may aid in lowering costs for users and/or responsible third parties, such as insurers, third party administrators, etc.

Various embodiments of systems, solutions, and/or methods that contain features the same as or similar to those discussed throughout may focus upon different types of users. For example, one system may be provided for and/or focused on users that do not have any form of insurance or responsible third party coverage for the procedures they are seeking. These may be users without any medical coverage at all, or users who have medical coverage, but that coverage does not cover the particular procedure or service that is desired (e.g., elective cosmetic surgery). In such a system, search results may be provided to the user regardless of any copayment, down payment, or other insurance or responsible third party payment coverage. In other systems, search results may be provided to the user that do consider one or more coverage amounts that may be paid on the user's behalf by insurance or a responsible third party, such as in an embodiment where the user or other entity provides the system with details about the coverage and/or plan that a user has.

In still other examples, a system may be provided that is for and/or focused upon users that have insurance or other responsible third party coverage for healthcare or other services (e.g., dental) and may tailor search results in response to that user's coverage or plan, as discussed in greater detail below. In one embodiment, an enterprise system may be provided by an insurance company or other responsible third party' company for users that they cover. Such a covered user may then sign into the system (e.g., using a login/password combination and/or account number that the user has obtained). Users having different insurance coverage and/or responsible third parties, may not be able to access an enterprise system for an insurance company or responsible third party company for which they do not have corresponding coverage. In an alternative embodiment, at sign-in, a user may choose from a user-interface element (e.g., a drop down box) and/or other user-interface element (e.g., a text box) a particular insurer or responsible third party with whom they are covered and/or provide the applicable account number. Upon sign-in, one or more features as discussed throughout may allow such user to search for a desired procedure and/or service, bundle, etc. and additionally see how much such would cost, this cost being reflective of the user's particular coverage. In order to accommodate such analysis on cost, the system would have access to one or more details about the user's coverage (e.g., either locally and/or via remote communication with another system and/or memory).

In still another example, a system (e.g., an enterprise system) may be provided that is for and/or focused upon employees or other personnel at an insurance company or other responsible third party coverage, rather than, or in addition, to end-users. For example, an enterprise system may utilize one or more details on the corresponding insurance company's or responsible third party company's covered users, such as their coverage amounts, copayment amounts, deductible amounts, etc. These details may be stored locally as part of the enterprise system and/or accessible via remote communication with another system and/or memory. Search results from the enterprise system may thus be tailored based upon a particular user's coverage and can allow for cost-savings by both the insurance company and the corresponding user, as exemplary′ shown below.

For example, if a user seeks to have a desired healthcare service performed and is covered by a particular insurance company, the user (or the user's doctor or other healthcare professional) may contact the user's insurance company to setup and/or initiate the process of obtaining the service performed under such coverage. If the insurance company uses the enterprise system having one or more of the features previously discussed, the insurance company may be permitted to run a search for the healthcare service and using the user's coverage details (e.g., deductible, copayment, etc.). Such search results may indicate a variety of possible options for having the service performed (and inclusive of other expenses, such as airfare, travel, etc.) that can be weighed against one another in an effort to save cost. For example, if the user is requesting an CAT-scan be performed, the cost for such a procedure may be cheaper in a foreign country, such as India, versus its cost to be performed in the United States. If the cost for the foreign country procedure, combined with the cost for any relevant travel accommodations, such as airfare, hotel, rental ear, etc. for the user is less expensive than the cost for the procedure in the United States, but without any necessary travel accommodations (e.g., if the United States location is close to the user's home address), the insurance company may choose to offer the overseas option to the user and agree to pay the relevant travel accommodations. In certain embodiments, other incentives may be offered to the user by the insurance company if the user agrees to undertake the additional “hassle” of traveling overseas for the procedure. Some such examples of these incentives may be lower insurance premiums, lower or lack of payment by the user for a copayment and/or deductible amount, etc. Indeed, any of a variety of incentives may be offered to the user. Thus, if accepted by the user, the insurance company may save money due to the Sower cost of the procedure inclusive of any travel accommodations and the user may save money due to the lack of or lower payments (e.g., deductible, copayment, etc.) required for him or her to pay out of pocket.

Insurance companies and/or other responsible third party companies may thus be incentivized to use and/or access such a system since it can help lower payments made through one or more of the various features discussed throughout. The system may be accessible by only insurance company or responsible third party personnel, accessible only by users, and/or accessibly by both insurance companies/responsible third party personnel and users, depending upon the desired configuration for focus for the particular embodiment. Providers of healthcare services may have incentive to offer lower prices, agree to cover travel accommodations, etc. in an effort to remain competitive with other healthcare service providers that are also or may sign up with the system, having the effect of promoting competition among service providers and aiding in the lowering of cost to insurance companies, responsible third parties, and/or users that utilize the system for locating desired healthcare procedures or services,

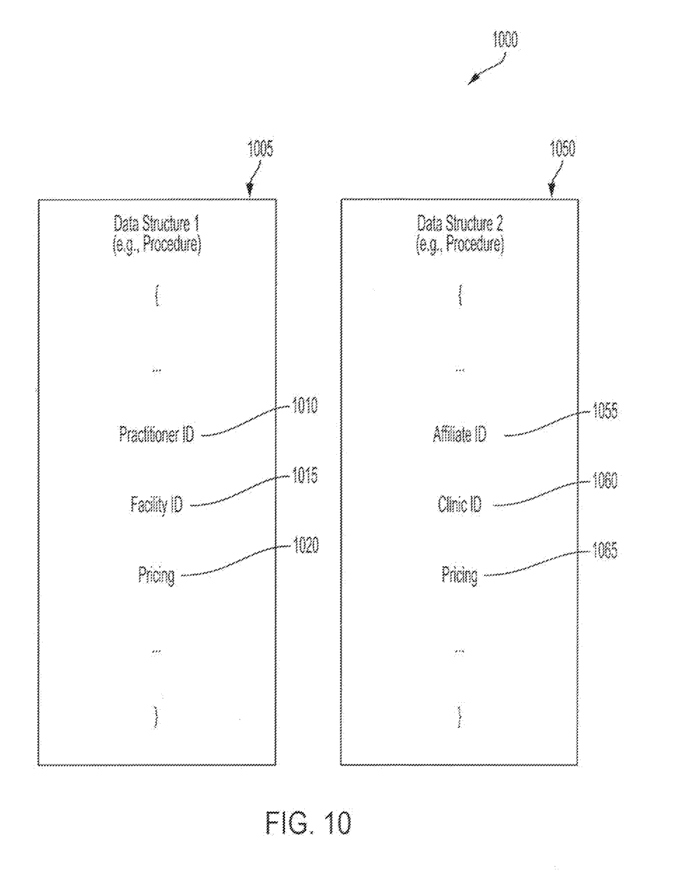

A variety of potential hardware and/or software setups may be used for creation of an apparatus or system that includes one or more of the features discussed throughout this application. For example, a system may utilize one or more computers or electronic devices or components, such as processors, memory, servers, networks or network devices (wired or wireless), etc., some of which are configured to execute software code and/or store or interact or manipulate data, such as digital data relating to medical procedures, practitioners, pricing, facilities, etc. In a system that uses a memory or storage of data, one or more data structures may be used for formatting or otherwise maintaining the data to aid in searching, locating, matching, or otherwise making determinations based upon the data stored. FIG. 10 illustrates a plurality of exemplary data structures 1000 for an apparatus or system for determining or requisitioning medical care. The apparatus system may include features that are the same as or similar to those discussed throughout this application.

A first data structure 1005 includes one or more variables, tags, flags, or elements that help maintain the data stored as part of or associated or linked with the first data structure 1005, for example, to allow or aid in subsequent look-up of the data or subsets of the data stored. The first data structure 1005 may correspond to a type of procedure (e.g., a medical procedure or service, such as a type of surgery or other treatment). Variables associated with the first data structure 1005, as discussed in greater detail below, may include or correspond with values providing additional information associated with that procedure (e.g., doctors who perform the procedure, where the procedures may take place, costs of the procedure, availability for scheduling of the procedure to be performed, other requirements associated with the procedure, such as age, sex, weight, or other requirements to be met before the procedure may be performed on a patient, etc.)

For example, a first variable 1010 may be stored as part of or associated with the first data structure 1005 and corresponds with an identification of a practitioner (e.g., a doctor or surgeon) who has been authorized or contracted as part of the system. A second variable 1015 may be stored as part of or associated with the first data structure 1005 and corresponds with a facility (e.g., an ambulatory surgery center, a hospital, medical clinic, surgery ward, etc.) where the procedure may be performed. A third variable 1020 may be stored as part of or associated with the first data structure 1005 and corresponds with pricing or a cost (e.g., an exact price, predefined price, pre-contracted price, etc. as discussed throughout this application) associated with the procedure. When data of the system is queried and/or searched, one or more of the variables and/or data structures may be queried to find appropriate results that match input received or otherwise determined (e.g., if a user requests or otherwise indicates a desire to search for available lung transplant procedures, the system may search or query one or more data structures concerning lung transplant procedures and/or search or query one or more variables associated with data structures to determine available or authorized doctors who perform such procedures, where such procedures may be performed, the cost of such a procedure, etc.

In another example, a second data structure 1050 includes one or more variables, tags, flags, or elements that help maintain the data stored as part of or associated or linked with the second data structure 1050, for example, to allow or aid in subsequent look-up of the data or subsets of the data stored. The second data structure 1050 may correspond to a type of auxiliary or affiliated service (e.g., a service or accommodation that may be desired in conjunction with a medical procedure, but is not the procedure itself, such as physical therapy or speech therapy services after a surgery has occurred). Variables associated with the second data structure 1050, as discussed in greater detail below, may include or correspond with values providing additional information associated with that service (e.g., vendors or affiliate companies who offer the service, where the service may take place, costs of the service, availability for scheduling of the service to be performed, other requirements associated with the service, such as age, sex, weight, or other requirements to be met before the services may be obtained by a patient, etc.)

For example, and as shown, a first variable 1055 may be stored as part of or associated with the second data structure 1050 and corresponds with an identification of an affiliate or company (e.g., a company, doctor, other professional, etc.) who has been authorized or contracted as part of the system to perform the service. A second variable 1060 may be stored as part of or associated with the second data structure 1050 and corresponds with a clinic (e.g., a hospital, medical clinic, or other building or location, etc.) where the services are available. A third variable 1065 may be stored as part of or associated with the second data structure 1050 and corresponds with pricing or a cost (e.g., an exact price, predefined price, pre-contracted price, etc. as discussed throughout this application) associated with the service. When data of the system is queried or searched, one or more of the variables and/or data structures may be queried to find appropriate results that match input received or otherwise determined (e.g., if a user requests or otherwise indicates a desire to search for physical therapy or if physical therapy is recommended or otherwise determined as potentially of interest to a user, the system may search or query one or more data structures concerning physical therapy services and/or search or query one or more variables associated with data structures to determine available or authorized affiliate services who perform such services, where such services may be performed, the cost of such a service, etc.

Although two exemplary data structures and corresponding variables have been explicitly illustrated in FIG. 10, any of a variety of possible options for storage of data concerning medical procedures, services, or other types of information, as discussed throughout this application, is possible in alternative embodiments. Greater, fewer, and/or different variables may be used in alternative embodiments. Data structures may be maintained in different manners in alternative embodiments. For example, rather than a data structure maintained by a procedure with corresponding variables associated with such a procedure (e.g., the first data structure 1005), an alternative data structure may be maintained by practitioner with corresponding variables associated with such a practitioner.

In one embodiment, a system may include medical data stored in a memory that is accessible via one or more processors, for example, over a network (wired or wireless, such as the Internet). The medical data may be stored as one or more data structures with a plurality of corresponding variables thereto or otherwise searchable. A user may attempt access of the system (e.g., via an authorization interface, such as a username and/or password), and determined to be approved to access the system based on the authorization information input. In certain embodiments, only particular doctors, clinics, procedures, etc. may be available for a given user (e.g., a user's particular authorization may only give the user access to treatments available from a subset of doctors). The user may further input a desired medical procedure they wish to obtain information about in order to potentially schedule. The system will search the medical data in the memory to locate the desired medical procedure with a match of the doctor that is available for the user. Pricing of the desired medical procedure may then be determined and/or output (e.g., transmitted, displayed, or otherwise provided) to the user. Based upon this information, the user may decide to schedule an appointment to have the procedure performed. In some embodiments, the user may be a direct user or consumer who is seeking medical care for themselves. In other embodiments, the user may be someone working on behalf of or at behest of the actual individual or patient who is seeking to undergo care (e.g., a nurse, doctor, or other administrative employee, or other individual, such as an employee of a hospital, insurance company, medical clinic, etc., who works with or for a potential patient by interfacing with the system for the patient and then relaying information received from the system to the patient).

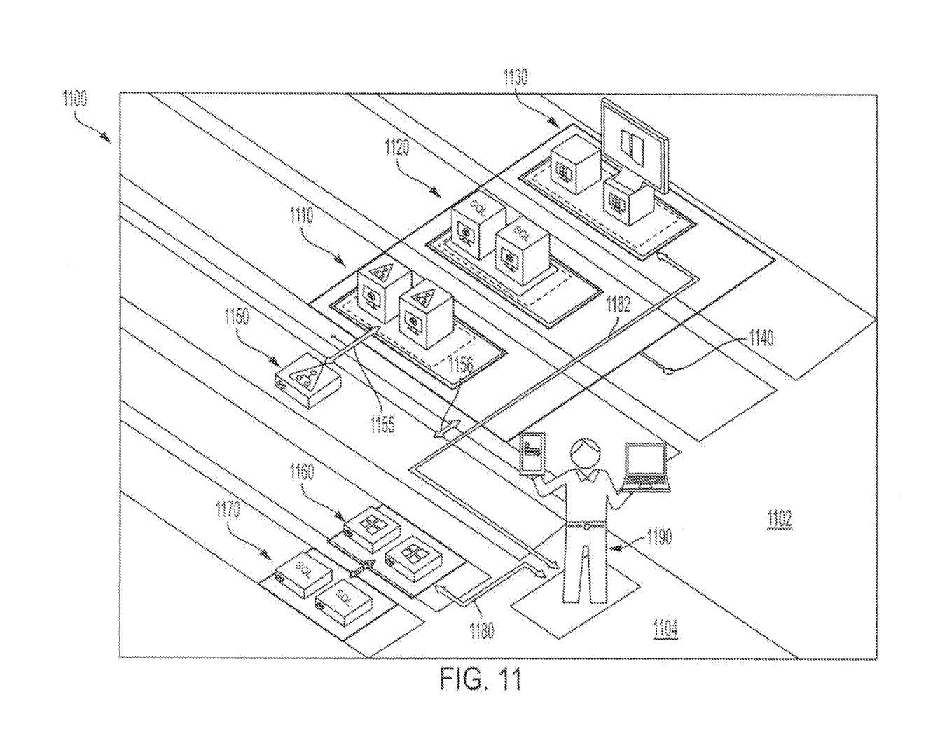

FIG. 11 illustrates an exemplary apparatus or electronic system 1100 for determining or requisitioning of medical care. The apparatus or electronic system 1100 may include features that are the same as or similar to those discussed throughout this application. The apparatus or system 1100 includes the interaction or interoperability of two infrastructures of electronic or computer equipment or components, a local infrastructure 1104 and a remote infrastructure 1102. A firewall, or other means of security, may be employed at the interface between the local infrastructure 1104 and the remote infrastructure 1102. The remote infrastructure 1102 may be a cloud-based infrastructure whereby equipment or components, as discussed in greater detail herein, are available and/or shared among a plurality of potential users or subscribers (e.g., businesses). For example, in one embodiment, the computing power of such equipment or components is thereby allocated based upon need at a given time among the plurality of potential users or subscribers to the cloud-based infrastructure rather than each potential user or subscriber having dedicated equipment or components that are available only for individual usage. Accordingly, a cloud-based infrastructure may allow for efficient usage of the computing power of such equipment or components. In an alternative embodiment, the remote infrastructure 1102 may not be cloud-based, but rather may be equipment or components that are specifically assigned to a particular user, subscriber, system, etc. and not shared among a plurality of different users, subscribers, systems, etc. The remote infrastructure 1102 may comprise a virtual network 1140 supporting login and access to remote users or machines substantially as if the equipment of the remote infrastructure 1102 was a local network.

The remote infrastructure 1102 may include a first tier of equipment 1110 for identifying and/or authenticating users or machines that attempt to connect (e.g., an “identity tier”). Such equipment in the first tier of equipment 1110 may be one or more virtual machines or other domain controllers. In one embodiment, upon identification and/or authentication of a user or machine on the one or more virtual machines, the data and/or applications available in the remote infrastructure 1102 may be made available for usage by the user or machine that has been identified and/or authenticated. If a user or machine is not properly identified and/or authenticated, access to such data and/or applications may be denied, e.g., for security purposes.

The remote infrastructure 1102 may also include a second tier of equipment 1120 for containing data or information (e.g., within a memory, database, etc.) of the remote infrastructure 1102 (e.g., a “data tier”). Such equipment in the second tier of equipment 1120 may be one or more servers, such as SQL servers. In one embodiment, the servers may utilize features that allow for access to the data or information (e.g., upon proper authorization by the first tier of equipment 1110) on an immediate or “always-on” basis to aid in the reduction of latency issues when retrieving and/or writing data to the memory or database. The memory or database may include a variety of possible information or data structures, organized in particular data structure format, for example, as discussed in greater detail throughout this application, such as the exemplary embodiments shown in FIGS. 10 and/or 12.

The remote infrastructure 1102 may also include a third tier of equipment 1130 for containing and/or executing applications or software code (e.g., an “app tier”). Equipment or components of the third tier of equipment 1130 may include computer systems (e.g., memory, processors, etc.). The applications or software code may communicate or otherwise access data or information from the second tier of equipment 1120. For example, an application may include an interface or backend for a user interface that retrieves information from and/or writes information to one or more equipment or components of the second tier of equipment 1120. In certain embodiments, duplicative and/or redundant applications may be stored and/or available for execution by the third tier of equipment 1130 (e.g., for redundancy in case of a failure or other undesirable interruption of service and/or for use in testing or scaling out of the application).

As discussed, in addition to the remote infrastructure 1102 having associated equipment and components, the local infrastructure 1104 may be configured to communicate 1156 (e.g., via secure and/or encryption-based communication protocols) with one or more of the equipment or components of the remote infrastructure 1102 (e.g., via a data bus or other path or paths allowing for the one-way and/or two-way communication of data or signals. In this fashion, certain aspects of the apparatus or electronic system 1100 for determining or requisitioning of medical care may be executed or accessed via only the local infrastructure 1104 (e.g., may be available with reduced latency, for example, for more time-sensitive operations), may be executed or accessed via only the remote infrastructure 1102 (e.g., may be stored in larger memory or databases or for which increased computing power is desired), and/or some combination thereof.

The local infrastructure 1104 may include a first tier of equipment 1150 for identifying and/or authenticating users that attempt to connect (e.g., an “identity tier”). The first tier of equipment 1150 may be or may include features that are the same as or similar to those previously discussed for the remote infrastructure 1102. For example, such equipment in the first tier of equipment 1150 may be one or more computers or machines that, upon identification and/or authentication of a user on the computer or machines, permit data and/or applications available in the local infrastructure 1104 may be made available for usage by the user that has been identified and/or authenticated. If a user is not properly identified and/or authenticated, access to such data and/or applications may be denied, e.g., for security purposes. One or more equipment or components of the first tier of equipment 1150 of the local infrastructure 1104 may be configured to communicate 1155 (e.g., by way of a data base or other communication method, such as a wired or wireless network connection, for example, via a Virtual Private Network) with one or more equipment or components of the first tier of equipment 1110 of the remote infrastructure 1102, for example, to permit a user or machine that is identified and/or authenticated in the local infrastructure 1104 and/or the remote infrastructure 1102 to automatically be identified and/or authenticated in the other. In this fashion, user experience may be benefited by allowing a user to more seamlessly interface with data and/or applications, whether they are stored and/or executed locally or remotely.

The local infrastructure 1104 may also include a second tier of equipment 1170 for containing data or information (e.g., within a memory, database, etc.) of the local infrastructure 1104 (e.g., a “data tier”). The second tier of equipment 1170 may be or may include features that are the same as or similar to those previously discussed for the remote infrastructure 1102. For example, such equipment in the second tier of equipment 1170 may be one or more servers (e.g., such as SQL servers) or other machines or components (e.g., connected or in communication with one another) for storage of digital data. In one embodiment, the machines or components may be expandable such that as data needs increase, additional machines or components may be added to facilitate the additional storage needs, such as via a data warehouse or connected machines or components. In various embodiments, machines or components of the second tier of equipment 1170 may be the same as, or different from, those of the second tier of equipment 1120 of the remote infrastructure 1104. The memory or database may include a variety of possible information or data structures, organized in particular data structure format, for example, as discussed in greater detail throughout this application, such as the exemplary embodiments shown in FIGS. 10 and/or 12.

The local infrastructure 1104 may also include a third tier of equipment 1160 for containing and/or executing applications or software code (e.g., an “app tier”). The third tier of equipment 1160 may be or may include features that are the same as or similar to those previously discussed for the remote infrastructure 1102. For example, equipment or components of the third tier of equipment 1160 may include computer systems (e.g., memory, processors, etc.). The applications or software code may communicate or otherwise access data or information from the second tier of equipment 1170. For example, an application may include an interface or backend for a user interface that retrieves information from and/or writes information to one or more equipment or components of the second tier of equipment 1170. In certain embodiments, duplicative and/or redundant applications may be stored and/or available for execution by the third tier of equipment 1160 (e.g., for redundancy in case of a failure or other undesirable interruption of service and/or for use in testing or scaling out of the application).

Users or machines 1190 for login to the system 1100 may access applications (e.g., user interfaces, web portals, other software applications, etc.) via communication or connection 1180 with the local infrastructure 1104 and/or communication or connection 1182 with the remote infrastructure 1102. In one embodiment, the user or machine 1190 may seamlessly interface with the system 1100 without requiring knowledge of or directing access to particular equipment or components of the system 1000 by way of the system 1100 automatically executing, retrieving, and/or storing data in either the local infrastructure 1104 and/or the remote infrastructure 1102 as needed. Users or machines 1190 may connect with the system 1100 via any of a variety of possible devices, such as laptops, desktop computers, mobile technology, such as smart phones or tablets, etc. For example, a software application existing as part of the remote infrastructure 1102 (e.g., the third tier of equipment 1130) and/or a software application existing as part of the local infrastructure 1104 (e.g., the third tier of equipment 1160) may be a web portal or other network or Internet-based application available for connection to and interaction therewith by one or more users or machines 1190, for example, to determine medical procedures, costs, practitioners, auxiliary services, etc. based upon data stored as part of the system 100 (e.g., via the second tier of equipment 1120 and/or 1170) and to provide or display such information or additionally determined or calculated information or data based upon such determinations based to the one or more users or machines 1190, as discussed in greater detail throughout this application. Although FIG. 11 illustrates one specific and exemplary embodiment of the system 1100, varying configurations and/or equipment setups may be used in an alternative embodiment for providing a system with one or more of the features discussed throughout this application.

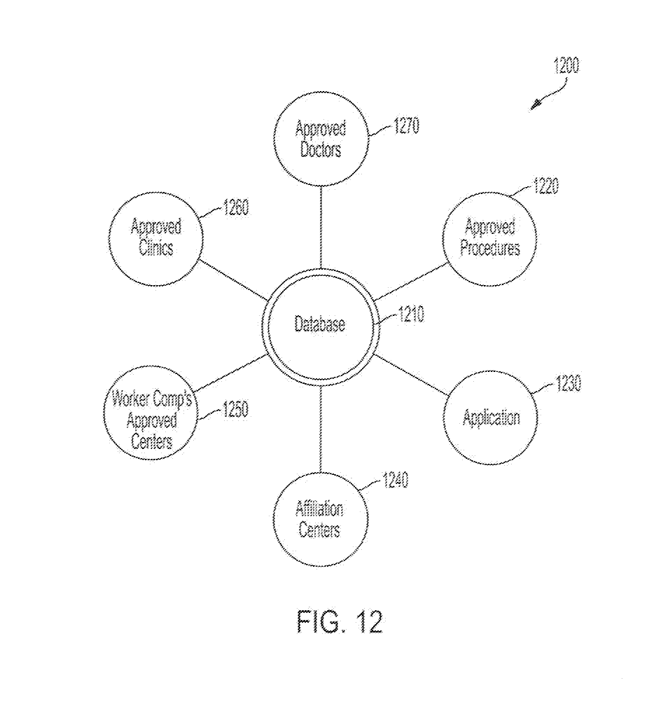

FIG. 12 illustrates an exemplary diagram 1200 of a plurality of data or information stored and used as part of an apparatus or system for determining or requisitioning of medical care. The apparatus or system may include features that are the same as or similar to those discussed throughout this application. For example, such data or information may be stored as part of a system for determining medical care, such as the system 1100 discussed for FIG. 11. In such an exemplary configuration, data structures or other storage of data having one or more features as discussed below for FIG. 12, may be included as part of a data tier of equipment (either local or remote infrastructure) for access by software applications (e.g., based upon prior identification or authorization of a particular user or machine). The diagram 1200 shows a database 1210 containing a variety of subcategories, variables, or structures of information related to medical care or other auxiliary services, as discussed in greater detail below. As discussed, the database 1210 may interface or with a software application 1230 or other executable code or system with access to the database 1210 for the purposes of reading and/or writing to the database 1210 and/or data connected or otherwise in communication with the database 1210.

The database 1210 may include and/or communicate with procedure data 1220 that relates or corresponds to available procedures (e.g., medical procedures). The procedure data 1220 may include medical procedures that have been approved by an administrator and/or otherwise incorporated into a system (e.g., system 1100 of FIG. 11) allowing for determination of medical care. For example, a system may be implemented whereby only medical procedures that have been previously allowed (e.g., pre-contracted) are included as part of the procedure data 1230 that is readable and/or writeable as part of the system. Until a particular medical procedure has been approved, it may not be included as part of the procedure data 1220 and/or may be flagged or otherwise indicated as part of the database 1210 or procedure data 1220 that it is not available for patient selection or use. Procedure data 1220 may, for example, be medical procedures, dental procedures, or any type of desired procedure of service in varying embodiments. The procedure data 1220 may be associated with or otherwise linked with the database 1210 and/or its other stored or accessible data, as discussed in greater detail herein.

The database 1210 may also include and/or communicate with doctor or practitioner data 1270 that relates or corresponds to available doctors or other practitioners (e.g., medical-based staff, surgeons, etc.). The doctor or practitioner data 1270 may include individuals in the medical field that that have been approved by an administrator and/or otherwise incorporated into a system (e.g., system 1100 of FIG. 11) allowing for determination of medical care. For example, a system may be implemented whereby only particular doctors or practitioners that have been previously allowed (e.g., pre-contracted) are included as part of the doctor or practitioner data 1270 that is readable and/or writeable as part of the system. Until a particular doctor or practitioner has been approved, it may not be included as part of the doctor or practitioner data 1270 and/or may be flagged or otherwise indicated as part of the database 1210 or doctor or practitioner data 1270 that it is not available for patient selection or use. Doctor or practitioner data 1270 may, for example, be medical doctors or surgeons, dental professionals, support staff, plastic surgeons, etc., or any type of desired individual or professional for performing a service for a consumer or patient in varying embodiments. The doctor or practitioner data 1270 may be associated with or otherwise linked with the database 1210 and/or its other stored or accessible data, as discussed in greater detail herein. For example, particular doctors or practitioners may be associated only with particular procedures included as part of the procedure data 1220.

The database 1210 may also include and/or communicate with clinic data 1260 that relates or corresponds to available clinics (e.g., medical, surgery, or hospital facilities, etc.). The clinic data 1260 may include those facilities or companies in the medical field that that have been approved by an administrator and/or otherwise incorporated into a system (e.g., system 1100 of FIG. 11) allowing for determination of medical care. For example, a system may be implemented whereby only particular clinics that have been previously allowed (e.g., pre-contracted) are included as part of the clinic data 1260 that is readable and/or writeable as part of the system. Until a particular clinic has been approved, it may not be included as part of the clinic data 1260 and/or may be flagged or otherwise indicated as part of the database 1210 or clinic data 1260 that it is not available for patient selection or use. Clinic data 1260 may, for example, be medical clinics, dental clinics, support or recovery clinics, etc., or any type of desired clinic or facility that allows for medical (or other) procedures to be performed for a consumer or patient in varying embodiments. The clinic data 1260 may be associated with or otherwise linked with the database 1210 and/or its other stored or accessible data, as discussed in greater detail herein. For example, particular clinics may be associated only with particular doctors or practitioners included as part of the doctor or practitioner data 1270 and/or for particular procedures included as part of the procedure data 1220.

The database 1210 may also include and/or communicate with worker's compensation data 1260 that relates or corresponds to available centers or facilities associated with worker's compensation claims. The worker's compensation data 1250 may include those centers or facilities that that have been approved by an administrator and/or otherwise incorporated into a system (e.g., system 1100 of FIG. 11) allowing for determination of medical care. For example, a system may be implemented whereby only particular centers or facilities involved in processing, managing, or otherwise involved in worker's compensation claims and/or treatment that have been previously allowed (e.g., pre-contracted) are included as part of the worker's compensation data 1250 that is readable and/or writeable as part of the system. Until a particular center or facility has been approved, it may not be included as part of the worker's compensation data 1250 and/or may be flagged or otherwise indicated as part of the database 1210 or worker's compensation data 1250 that it is not available for patient selection or use. Worker's compensation data 1250 may, for example, clinics, offices, professionals, or any type of desired center or facility involved in the process of facilitating, managing, or treating patients undergoing worker's compensation claims or matters. The worker's compensation data 1250 may be associated with or otherwise linked with the database 1210 and/or its other stored or accessible data, as discussed in greater detail herein. For example, particular centers or facilities may be associated only with particular medical clinics as part of the clinic data 1260, particular doctors or practitioners included as part of the doctor or practitioner data 1270 and/or for particular procedures included as part of the procedure data 1220.

The database 1210 may also include and/or communicate with affiliation or auxiliary service data 1240 that relates or corresponds to available centers, clinics, facilities, or professionals involved in affiliated, complementary, or other auxiliary services or treatments to a user's or patient's primary procedure (e.g., recovery centers, physical therapy centers, speech facilities, etc.). The affiliation or auxiliary service data 1240 may include those centers, or facilities, or individuals (e.g., professionals, therapists, etc.) that that have been approved by an administrator and/or otherwise incorporated into a system (e.g., system 1100 of FIG. 11) allowing for determination of medical care. For example, a system may be implemented whereby only particular centers, facilities, or individuals involved in providing affiliated or auxiliary services or procedures to a primary procedure that have been previously allowed (e.g., pre-contracted) are included as part of the affiliation or auxiliary service data 1240 that is readable and/or writeable as part of the system. Until a particular center, facility, or individual has been approved, it may not be included as part of the affiliation or auxiliary service data 1240 and/or may be flagged or otherwise indicated as part of the database 1210 or affiliation or auxiliary service data 1240 that it is not available for patient selection or use. Affiliation or auxiliary service data 1240 may, for example, clinics, offices, professionals, or any type of desired center or facility involved in the setup, management, and/or provision of additional products, services, or other treatments, either before or after a patient's other procedure or service. The affiliation or auxiliary service data 1240 may be associated with or otherwise linked with the database 1210 and/or its other stored or accessible data, as discussed in greater detail herein. For example, particular affiliated or auxiliary centers, facilities, or individuals may be associated only with particular worker's compensation centers included as part of the worker's compensation data 1250, particular medical clinics as part of the clinic data 1260, particular doctors or practitioners included as part of the doctor or practitioner data 1270 and/or for particular procedures included as part of the procedure data 1220.

By establishing a database (e.g., the database 1210) and/or multiple linked databases or other forms of data storage in memory, any of approved procedures, doctors, clinics, worker's compensation centers, affiliation centers, and/or or other types of facilities, services, individuals, treatments, etc. may be algorithmically or otherwise searched and particular options determined and/or displayed for a given user or a given patient, as discussed throughout. For example, a prospective patient, or individual working for or at behest of a prospective patient, may login to a system (e.g., system 1100 of FIG. 11) and interface with a user-interface or other software application to input desired patient characteristics or desires (e.g., treatment or procedure desired, geographic location, maximum price, preferred doctor, etc.) The system may then search information available in its database and/or multiple databases, to determine matches or options for the prospective patient. For example, in one algorithm, if a user of the system indicates that price is of utmost importance, one or more matches of the user's desired procedure that have a competitive price, such as a lowest price (or set of competitive prices) and/or prices at a lower range of the prices found in the system (e.g., regardless of geographic location of the doctor or clinic with respect to the geographic location of a residence of the prospective patient) may be displayed in priority to and/or to the exclusion of other matches where prices may be higher in value. Any of a variety of possible algorithms or other criteria for governing the search of the database, either based upon user preference input or without any user preference input, may be used in alternative embodiments.

In certain embodiments, a precise, exact, or specific price may be displayed with one or more of the associated options returned by the system. For example, the price of a particular procedure may be stored as part of the database 1210 and/or as part of its linked access to other data. Different exact pricing may be available depending on the procedure, the doctor, the clinic, the worker's compensation center, the affiliation center, and/or any combination thereof or of other data stored as part of the database 1210 or its linked access to other data. In one embodiment, precise pricing may be established as part of the allowance of a particular element (e.g., facility or individual) as part of the system. For example, in order to be approved and included as part of the system, a doctor and/or clinic may be required to agree to perform a particular procedure or treatment at a predefined, exact price. This price may be established by the administrator and/or owner of the system. This exact price may be the same or consistent for the same procedure or treatment across the system, regardless of the particular individual (e.g., doctor) and/or facility (e.g., hospital, medical clinic, etc.) that is involved in providing the procedure or treatment. Thus, in such an embodiment, procedures or treatments in such a system may be standardized in pricing. In an alternative embodiment, different individuals (e.g., doctors) and/or different facilities (e.g., an ambulatory surgery center, hospital, medical clinic, etc.) may be permitted to have different exact pricing established and still be allowed and/or authorized to be included as part of the data in the system that is searchable for prospective patients. In still other embodiments, the database 1210 and/or its linked access to other data may not be gated by any particular administrator and/or owner of a system, but rather freely allow individuals, facilities, etc. to manually include their own provided services and pricing, without first undergoing an authorization process to be included as part of the system.

Pricing may also or alternatively be established via bundling of services. For example, while a particular procedure may incorporate an exact, specific, or precise price that can be determined through searching through or interactions with the system or apparatus, bundled pricing on a plurality of procedures, services, or other products may also or alternatively be determinable. For example, if a patient, or individual or navigator working with or on behalf of a patient, interfaces with a system for determining possible doctors, hospitals, clinics, etc. for receiving the desired medical care for the patient, ancillary expenses or other services or products (e.g., travel costs like airfare or rental cars, pharmaceuticals or other medicines, medical products such as braces or wheelchairs, etc.) may be bundled with the desired medical care for a bundle price. The bundle price may be an exact, specific or precise price that encompasses all of the services or products bundled together and/or may be a different (e.g., lower) price than would otherwise be obtained by adding together the exact price associated with each individual service or product that is bundled. In one example, a patient who desires cardiac surgery may require to travel to a geographic location that is far from their residence and could thus select a bundle pricing that includes all or some of the travel-associated costs for traveling to the hospital, clinic, or other facility in order to see the doctor or surgeon and have the procedure performed. Any of a variety of possible services or products (e.g., physical therapy, speech therapy, counseling, medical products, home products, etc.) may be available for bundling by the system.

In one embodiment, an apparatus or system may be configured for sign-in by two types of users. A first type of user may be a company, organization, individual, etc. who uses the apparatus or system to search for and/or determine medical care, either for themselves or on behalf of others. For example, the first type of user might be a company or organization that provides its employees or contractors with healthcare or medical benefits. Rather than (or supplemental to) use of traditional healthcare methods that a company may pay for, whereby a medical insurance company is engaged for processing medical claims of its employees, the company or organization may contract to be part of or have access to the apparatus or system. Based on the information (e.g., exact pricing for medical procedures, etc.) provided by the apparatus or system that the company or organization has access to as a result of its participation with the apparatus or system, an employee or contractor of that company or organization (or the company or organization may choose on behalf of the employee) to receive the desired medical care and have the corresponding costs of the medical care paid in accordance with the apparatus or system information, rather than going through a traditional insurance company.

For example, a company with access to the information of the apparatus or system may determine that the cost associated with a medical procedure may be less expensive via direct payment of the exact price determined from access to the apparatus or system (e.g., a price that the apparatus or system has already pre-contracted in its database with one or more doctors, hospitals, clinics, etc.). In such a case, the company may choose and/or let the employee who is having the procedure performed choose, whether direct payment of the less expensive price is desired, or if a traditional payment method for the procedure is desired (e.g., having the employee enrolled in a medical insurance plan where a portion of the employees paycheck is withheld to pay for costs associated with the enrollment of the employee in a particular medical plan through the insurance company or other medical plan provider, submittal of the claim to the insurance company or medical plan provider, and paying any further associated costs, such as copayments, deductibles, etc.). Participation in such an apparatus or system may be desirable for companies or organizations where medical procedures are common (e.g., athletic or sports companies or leagues) and/or for any companies, organizations, groups, etc. where cost savings may be achieved through direct payment of less expensive, pre-contracted pricing for procedures as opposed to traditional medical care plans and the corresponding overhead of managing such plans for employees.

In one embodiment, in order to have access, the first type of user may pay a fee (e.g., a monthly, annual, or otherwise subscription or payment in order to have access to the medical procedure data and associated exact pricing that is pre-contracted to by the various doctors or clinics that are allowed or authorized to participate as part of the apparatus or system). In another embodiment, the first type of user may not have to pay any fee for access. In such an embodiment, the apparatus or system may receive revenue from the doctors, hospitals, clinics, etc. that are pre-contracted to participate in the system (e.g., a fee to be included in the apparatus or system and returned as part of search results). In another embodiment, the apparatus or system may receive revenue by taking a fee (e.g., a flat rate, a percentage, etc.) when a service looked up via the apparatus or system is performed and paid for (e.g., if an individual has a medical procedure performed in accordance with the use of the apparatus or system to locate the doctor/hospital and its pre-contracted pricing, then a portion of the payment for that medical procedure would go to the administrator or owner of the apparatus or system).

A second type of user that of the apparatus or system may be administrators or other individuals with permissions to access features of the apparatus or system that relate to status, management, maintenance, or other information relating to the first type of user. For example, an administrator of the system may be permitted to add new companies or organizations (e.g., the first type of user) who wish to have access to the apparatus or system.

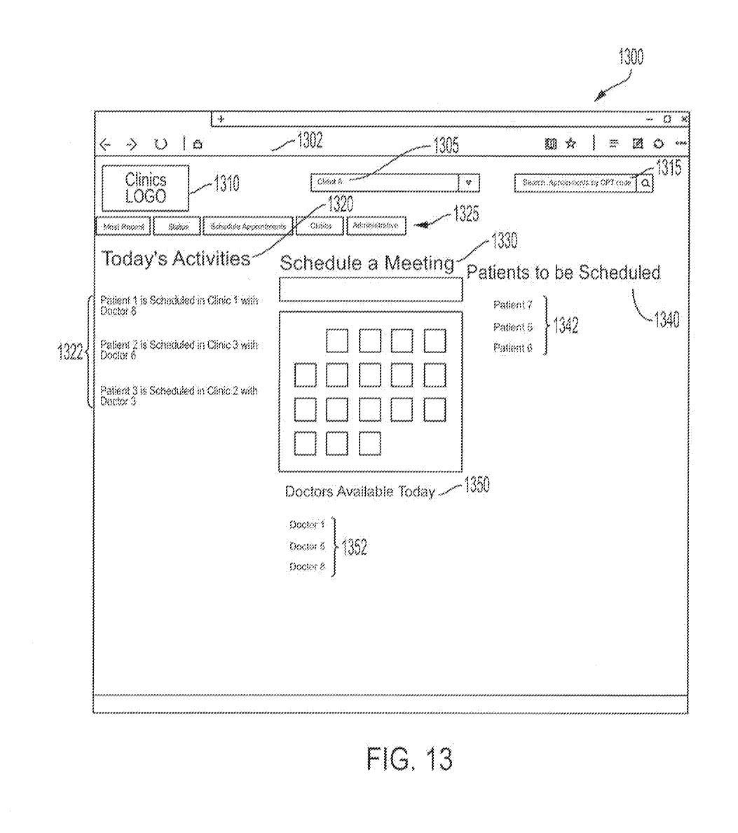

FIG. 13 illustrates a display 1300 of a user-interface for an administrator of an apparatus or system for determining or the requisitioning of medical care. The user-interface 1300 may be as part of a webpage or web portal that is accessible over a network (e.g., the Internet) by directing a web browser to a particular address 1302 (e.g., a URL) and entering corresponding authentication (e.g., username and/or password information). The system or software application incorporating the user-interface 1300 may include hardware, equipment or components configured to identify and/or authorize potential users that are attempting access before allowing such potential users access to further applications and/or data, for example, as previously discussed. In an alternative embodiment, software other than a web browser may be used for accessing and/or confirming a user's access to the system, for example, software that is provided as part of the system itself

The user-interface 1300 may include a manipulatable element 1305 (e.g., a drop-down box) that allows an administrator accessing the system to select a particular client or user that has been setup as an authorized entity to that is part of the system (e.g., a company, an organization, a medical group, etc.). Selection of a particular client may update the data that is retrieved for display upon the display 1300. For example, as discussed in greater detail below, particular activities, doctor availability, and/or scheduling of patients may be different for different users or clients of the system. Accordingly, selection of different clients via manipulatable element 1305 may result in different data or information being shown. A graphic, logo, or other identifier 1310 may be displayed upon the selection of a particular client via element 1305 (e.g., a company logo associated with the client selected).

To aid in display of desired information, a searching element or feature 1315 may allow the administrator to search for a particular appointment scheduled with respect to a client via medical procedure or treatment (e.g., via Current Procedural Terminology or “CPT” code). In varying embodiments, the searching element or feature 1315 may allow for searching of varying types of information, including by patient name, doctor name, date, etc. A list of selectable tabs 1325 or other means of navigation allow an administrator to conveniently navigate among various information available with respect to the client selected via the manipulatable element 1305, such as most recent activity, status, appointment scheduling, available clinics to the client, or other administrative tasks (e.g., assignment of available doctors, facilities, procedures, payment or invoicing status, etc.)

A section 1320 of the display 1300 corresponds to a list of the daily activities related to the client and includes further information 1322 associated with those daily activities (e.g., which patients associated with the client are scheduled with particular doctors, at particular facilities or clinics, etc. A section 1340 of the display 1300 corresponds to a list of patients of the client that still need to be scheduled for a procedure and includes further information 1342 associated with such scheduling (e.g., which patients associated with the client have indicated a need or desire for a particular medical procedure, doctor visit, treatment, etc., but who do not have a scheduled appointment or time to have such performed.) Similarly, a section 1350 of the display 1300 corresponds to a list of doctors with availability (e.g., on that particular day, or on other dates as desired in alternative embodiments) for scheduling with a patient and includes further information 1352 associated with such availability (e.g., doctor names, times of day, etc.). A section 1330 of the display allows for scheduling of a patient of the client with a particular doctor, for example by providing a user-interface element, such as a calendar that allows for selection of a particular date or time to arrange such an appointment.

Although the display 1300 specifically illustrates particular user-interface elements and/or configuration of data for a user (e.g., an administrator) of the system, alternative elements and/or orientations and/or data may be used in an alternative embodiment. Moreover, in certain embodiments, the manipulatable element 1305 may be removed for display to a particular client (as opposed to an administrator of the system) to allow such client to view patient and doctor scheduling information and/or organize appointments. Any of a variety of possible user interfaces may be used to aid in allowing a client and/or prospective patients associated with a client identify, search, and/or see information concerning medical procedures, practitioners, clinics, affiliated services, etc. through interaction with hardware and/or software interfaces of an apparatus or system having certain features as disclosed throughout this application.

The previous description of the disclosed examples is provided to enable any person of ordinary skill in the art to make or use the disclosed methods and apparatus. Various modifications to these examples will be readily apparent to those skilled in the art, and the principles defined herein cars be applied to other examples without departing from the spirit or scope of the disclosed method and apparatus. The described embodiments are to be considered in all respects only as illustrative and not restrictive and the scope of the invention is, therefore, indicated by the appended claims rather than by the foregoing description. All changes which come within the meaning and range of equivalency of the claims are to be embraced within their scope. Skilled artisans can implement the described functionality in varying ways for each particular application, but such implementation decisions should not be interpreted as causing a departure from the scope of the disclosed apparatus and methods. The steps of the method or algorithm can also be performed in an alternate order from those provided in the examples.

What is claimed is:

1. A system to requisition medical care comprising:
a memory configured to store medical data, the medical data comprising one or more data structures including:
a pricing variable that defines an exact price for a medical procedure, and
a practitioner variable that defines a practitioner to perform the medical procedure; and
one or more processors connected with the first memory via a network and configured to:
receive authorization data,
communicate with the first memory via the network to identify approved practitioner data based upon the authorization data;
receive request data,
communicate with the first memory via the network to locate a medical procedure in the medical data based upon the request data and matching the practitioner variable with the approved practitioner data, and
communicate with the first memory via the network to output an exact price for the medical procedure based upon the pricing variable.

2. The system of claim 1 wherein the network is a cloud-based plurality of servers.

3. The system of claim 1 further comprising a second memory remote from the first memory and configured to communicate with the first memory via wireless signal transmission.

4. The system of claim 3 wherein the second memory and the first memory are synchronized.

5. The system of claim 4 wherein the synchronization occurs on a daily basis.

6. The system of claim 1 wherein the receipt, by the processor of authorization information from the user occurs via a virtual machine.

7. The system of claim 6 wherein the virtual machine is accessed via a web portal over the Internet.

8. The system of claim 1 wherein the one or more data structures of medical data further comprises:
an affiliate variable that defines a type of affiliate service, and
an affiliate pricing variable that defines an exact price for the type of affiliate service.

9. The system of claim 8 wherein the one or more processors are further configured to:
communicate with the first memory via the network to locate an affiliate service in the medical data.

10. The system of claim 9 wherein the one or more processors are further configured to:
communicate with the first memory via the network to output a bundled price for the medical procedure bundled with the affiliate service,
wherein the bundled price is less than the exact price for the medical procedure added to the exact price for the affiliate service.
